(19) United States
(12) Patent Application Publication
(10) Pub. No.: US 2024/0366271 A1
(43) Pub. Date: Nov. 7, 2024

(54) INTEGRALLY FORMED MEDICAL DEVICES

(71) Applicant: 3DMorphic Pty Ltd, Matraville (AU)

(72) Inventor: William C.H. PARR, Matraville (AU)

(21) Appl. No.: 18/686,289

(22) PCT Filed: Aug. 25, 2022
(86) PCT No.: PCT/AU2022/051021

§ 371 Date: Feb. 23, 2024

(30) Foreign Application Priority Data
Aug. 25, 2021 (AU) 2021221798

(51) Int. Cl. A61B 17/70; A61B 17/00; A61F 2/30; A61F 2/44; A61F 2/46

(52) U.S. Cl. CPC A61B 17/7059; A61F 2/30942; A61F 2/447; A61F 2/4601; A61B 2017/00526; A61F 2002/3071; A61F 2002/30985

(57) ABSTRACT

Disclosed herein are integrally formed medical devices and methods relating thereto. One embodiment provides an integrally formed medical device comprising: a substantially trapezoidal body portion having at least one surface that is contoured to fit an anatomy of a patient; and a graft window defining a vertical aperture through said body portion, such that said body portion defines a cage.

### RELATED APPLICATION

This application is related to Australian Patent Application No. 2021221798 titled “Integrally formed medical devices”, filed 25 Aug. 2021 in the name of 3DMorphic Pty Ltd, the entire content of which is incorporated by reference as if fully set forth herein.

### TECHNICAL FIELD

The present disclosure relates to medical devices. In particular, the present disclosure relates to spinal devices, including interbody and corpectomy/vertebrectomy devices.

### BACKGROUND

Spinal instrumentation relates to the use of instruments in surgical procedures on the spine. There are many different types, shapes, and sizes of instruments designed to treat different spinal disorders and conditions and such instruments include rods, plates, screws, and cages. To minimise adverse reactions from the patients, the instruments, also referred to as medical implants or medical devices, are typically manufactured from biocompatible materials, such as titanium, titanium-alloy, stainless steel, or non-metallic materials (e.g., calcium phosphates, calcium silicates, ceramics or polymers, such as methacrylates, polyether ether ketone (PEEK), polyether ketone ketone (PEKK), and other members of the polyaryletherketone (PAEK) polymer family).

Spinal interbody devices are designed to be placed between adjacent vertebrae and are used in the treatment of discogenic spinal pathology. Such spinal interbody devices aim to restore lost disc height, thereby preventing osteoarthritic bone-bone grinding of adjacent vertebral body endplate bone. Restoring lost disc height through the insertion of a spinal interbody device between vertebrae can also decompress neural structures, such as nerve roots and the spinal cord.

Spinal interbody devices can be used to stabilise the instrumented level (i.e., the region of the spine on which medical devices are used) to relieve the dynamic components contributing to clinical symptoms of a patient. Dynamic components may include, for example, ‘pinching’ of soft tissue structures, such as nerve roots. Spinal interbody devices also provide stable biomechanical conditions suitable for growth of bone on and into the implanted device, known as osseointegration. Osseointegration can contribute to interbody bone fusion and aid in long term stability and symptom relief.

Further, spinal interbody devices can be used to re-align vertebrae to treat pathologies resulting from excessive coronal plane spine curvature (scoliosis) or lack of sagittal plane spine curvature (lordosis).

A corpectomy/vertebrectomy is a surgical procedure that removes a portion of one or more vertebral bodies and adjacent discs. Spinal corpectomy/vertebrectomy devices are used to treat conditions in which the cord is compressed and/or the vertebral body has become unstable and/or unable to function mechanically in supporting the anterior column of the spine.

Causes of cord compression necessitating corpectomy can include: ossification of the posterior longitudinal ligament (PLLO); haematoma; burst fractures of the vertebral body with fracture fragments moving in a posterior direction (into the spinal canal); and tumour.

Causes for loss of mechanical function of the vertebral body can be loss of bone mineral density, and thereby stiffness, as a result of: osteopenia; osteoporosis; traumatic fracture; bone lysis resulting from infection, tumour, or due to, for example, wear debris from implanted devices in the area.

Corpectomy devices aim to stabilise the treated level by spanning between the vertebral bodies superior and inferior to the pathological vertebral body and can be used to restore spinal element (vertebrae) alignment. The devices also aim to create stable conditions suitable for osseointegration and bone fusion.

Current medical implants require multiple processing steps. There is a technical and manufacturing need to be able to produce a medical implant with a reduced number of post-production steps before being ready for use.

### SUMMARY

The present disclosure relates to integrally formed medical devices and methods for manufacturing such devices.

A first aspect of the present disclosure provides an integrally formed medical device comprising:
a substantially trapezoidal body portion having at least one surface that is contoured to fit an anatomy of a patient; and
a graft window defining a vertical aperture through the body portion, such that the body portion defines a cage.

A second aspect of the present disclosure provides a method of producing a medical device, comprising the steps of:
importing a first digital model of a medical device to fit an anatomy of a patient, wherein the medical device has a substantially trapezoidal body portion and a graft window defines a vertical aperture through the body portion, such that the body portion defines a cage;
warping at least one surface of the digital model of the medical device to produce a second digital model of a device; and
utilising an additive manufacturing machine to manufacture the medical device, based on the second digital model of the device.

In some embodiments, the first digital model of the medical device is a triangulated vertex boundary representation suitable for 3D printing, and wherein the warping is performed by:
importing a third digital model of a target shape associated with the anatomy of the patient;
determining a warping interpolation function based on relative positions of a set of source points associated with the first digital model and relative positions of the same points projected to the target shape; and
applying the warping interpolation function to all vertices of the medical device to generate the second digital model of the device.

In some embodiments, the anatomy of the patient is associated with a statistical shape model suited to the patient.

In some embodiments, the anatomy of the patient is associated with a model of the anatomy from a specific patient, wherein the first digital model fits the model of the patient-specific anatomy, so as to produce a patient-specific medical device.

In some embodiments, the warping is performed using at least one parametric computer aided design (CAD) methods based in Bezier surfaces, Non-uniform rational B-spline surfaces, or Catmull-Clark subdivision of surfaces.

In some embodiments, the additive manufacturing machine is a 3D printer that manufactures the patient-specific device using titanium or another biocompatible material.

According to another aspect, the present disclosure provides an apparatus for implementing any one of the aforementioned methods.

According to another aspect, the present disclosure provides a computer program product including a computer readable medium having recorded thereon a computer program that when executed on a processor of a computer implements any one of the methods described above.

Other aspects of the present disclosure are also provided.

### BRIEF DESCRIPTION OF THE DRAWINGS

One or more embodiments of the present disclosure will now be described by way of specific example(s) with reference to the accompanying drawings, in which:

FIGS. 1A and 1B relate to the use of a patient-specific spinal device to restore the interbody and neuroforaminal height in a patient having a collapsed/pathological disc;

FIGS. 2A to 2E illustrate various anterior cervical discectomy and fusion (ACDF) devices;

FIGS. 3A and 3B provide a comparison between the fit of a generic ACDF device and that of an ACDF device contoured to fit a patient anatomy;

FIGS. 4A and 4B provide a comparison between a generic ACDF device and an ACDF device contoured to fit a patient anatomy;

FIGS. 5A and 5B provide a comparison of the contact surface area and penetration of devices through the vertebral body endplates between an ACDF device contoured to fit a patient anatomy and a generic shape, flat-sided ACDF device;

FIG. 6 is an intra-operative x-ray of an ACDF device;

FIG. 7A shows an Anterior Lumbar Interbody Fusion (ALIF) device;

FIG. 7B is an enlarged view of the device of FIG. 7A;

FIGS. 8A to 8C show small, medium, and large variations of Anterior Lumbar Interbody Fusion (ALIF) devices;

FIG. 9 is a lateral (sagittal plane) view of a cervical corpectomy/vertebrectomy device;

FIG. 10 is a superior (axial plane) view of a cervical corpectomy/vertebrectomy device;

FIGS. 11A to 11G show a cervical corpectomy/vertebrectomy device 1100 viewed from various viewpoints;

FIG. 12 is an anterior (coronal plane) view of a lumbar corpectomy/vertebrectomy device;

FIGS. 13A to 13F show a lumbar corpectomy/vertebrectomy device with holes for integral screws;

FIG. 14 is an anterior right lateral view of a lateral approach interbody device;

FIG. 15 is an anterior right lateral view of a lateral approach interbody device;

FIG. 16 is an anterior left lateral view of the lateral interbody device shown in FIG. 15;

FIG. 17 is an anterior view of a lateral interbody device with one integral screw fixation screw;

FIG. 18 is a simulated anterior-posterior (AP) x-ray of a lateral interbody device; and

FIG. 19 is a schematic representation of a computing device on which software for warping a digital model of a medical device may be practised.

Method steps or features in the accompanying drawings that have the same reference numerals are to be considered to have the same function(s) or operation(s), unless the contrary intention is expressed or implied.

### DETAILED DESCRIPTION

The present disclosure provides embodiments of medical devices, particularly spinal interbody devices, that can be manufactured by additive manufacturing, also known as 3D printing or rapid prototyping. The medical devices are produced as integral units using additive manufacturing techniques, so as to require minimal post-processing before surgical use. Alternative embodiments of medical devices contemplated herein may be produced using other manufacturing processes, such as Computer Numerical Control (CNC) machining.

Some embodiments relate to integrally formed medical devices that include a substantially trapezoidal body portion having at least one surface that is contoured to fit an anatomy of a patient. The “anatomy of a patient”, also referred to herein as “patient anatomy”, may relate broadly to a part of the human body, such as a generic bone, particular bone, intervertebral spacing, or the like. In such cases, the resultant medical devices are suitable for use in a range of patients. For example, a medical device may be a spinal device having at least one surface contoured to fit in the space between vertebrae and suitable for a range of human patients. In such an example, the anatomy of the patient relates to the intervertebral spacing based on a model of a human body.

Alternatively, the anatomy of a patient may be specific to an individual patient, in which case the medical device is patient-specific. Using the same example of a spinal device, at least one surface contoured to fit in the space between vertebrae in the patient-specific implementation would be based on the particular anatomy of the individual patient.

Where the patient anatomy relates generally to a part of the human body, the patient anatomy may be associated with a statistical shape model suited to the relevant part of the human body. The statistical shape model may have a set of associated model parameters to make the medical device suitable for a particular type of patient or demographic, or a combination thereof. The parameters may relate to the device and include, for example, device length, device width, device height, contact surface area, device volume, surface curvature, and intended site (spine level) for use. The parameters may also relate to demographics, which may include patient height, patient weight, patient age, type of pathology, and the like. By changing one or more of the parameters, a set of generic medical devices may be produced for different demographic profiles, as defined by the parameters. Where the patient anatomy is specific to an individual patient, the patient anatomy may be associated with a model of the anatomy of that individual patient, such as may be derived from imaging and the like.

Some embodiments relate to integrally formed medical devices that include a substantially trapezoidal body portion having at least one surface that is contoured to fit an anatomy of a patient. As described above, the anatomy of a patient may be broadly defined, relating to a part of the human body to which the medical device is to interface, or alternatively may be patient-specific. The devices also include a graft window defining a vertical aperture through said body portion, such that said body portion defines a cage. In some implementations, the medical devices are integrally formed using an additive manufacturing process, such as 3D printing, from titanium, titanium alloys (such as Ti₆Al₄V), or other biocompatible materials.

In order to produce integrally formed devices, one or more of the embodiments described herein may be designed using one or more of the methods described in Australian Provisional Patent Application No. 2016900216 titled “Method and system for designing and fabricating a customised device” and filed 25 Jan. 2016 in the name of 3DMorphic Pty Ltd and International Patent Application No. PCT/AU2017/050056 titled “Method and system for designing and fabricating a customised device” and filed 25 Jan. 2017 in the name of 3DMorphic Pty Ltd, the content of each of which is incorporated herein by reference as if fully set forth herein.

In particular, surfaces of medical implants may be warped or contoured to fit patient anatomy using one or more of the methods described in those applications. Other methods of producing devices, such as various parametric CAD methods based in Bezier surfaces, Non-uniform rational B-spline surfaces, or Catmull-Clark (or other styles) of subdivision surfaces may also be practised.

The present disclosure provides a method for producing a medical device that includes importing a first digital model of a medical device to fit an anatomy of a patient, wherein the medical device has a substantially trapezoidal body portion and a graft window defines a vertical aperture through the body portion, such that the body portion defines a cage. The method warps at least one surface of the first digital model of said medical device to produce a second digital model of a medical device and then utilises an additive manufacturing machine to manufacture the medical device, based on the second digital model of the medical device.

In some implementations, the medical device is a patient-specific device.

In some implementations, the first digital model of the medical device is a triangulated vertex boundary representation suitable for 3D printing.

In some implementations, the warping of the at least one surface of the first digital model of the medical device is performed by: importing a third digital model of a target shape associated with the anatomy of the patient, and determining a warping interpolation function based on relative positions of a set of source points associated with the first digital model and relative positions of the same points projected to the target shape. The method then applies the warping interpolation function to all vertices of the medical device to generate the second digital model of the patient-specific device, which is subsequently used to print the medical device.

In some implementations, the target shape associated with the anatomy of the patient relates to a normalised model of a part of the human anatomy corresponding to a location within the patient to which the device is to be implanted. In some implementations, the target shape associated with the anatomy of the patient is specific to the anatomy of that patient.

In some arrangements, the third digital model of the target shape is acquired by scanning the anatomy of the patient and manipulating the scans to acquire the target shape.

In other implementations, the warping is performed using at least one parametric computer aided design (CAD) methods based in Bezier surfaces, Non-uniform rational B-spline surfaces, or Catmull-Clark subdivision of surfaces.

Integrally formed (i.e., 1-piece) devices disclosed herein increase overall manufacturing speed and efficiency by reducing the number of post-processing steps involved, for example, in machining or adding additional features. Further integrally formed devices are safer, as by being 1-piece devices there is less chance of device delamination, parts disassociation, part-part wear, and inter-part corrosion reactions. Integrally formed devices also provide improved quality of traceability via an optional identifier incorporated within the device, such as through additive printing, embossing, or debossing.

Some embodiments relate to medical implants having one or more integrated alignment aids that assist intra-operative workflow efficiency. Attributes of the alignment aids enable a user, such as a surgeon, to orient the device quickly and accurately during implantation as well as assisting in intra and post-operative radiographic alignment assessment. Such attributes may include, for example, one or more of the size, shape, positioning, and orientation of an alignment aid relative to the device or with reference to other alignment aid on the device.

Alignment aids may take many different forms, including writing, arrows, symbols, and the like. In some embodiments, the alignment aid is a set of one or more apertures. In particular, in some embodiments the medical devices include alignment aids in the form of one or more triangular windows. Alignment aids in the form of one or more triangular windows have particular advantages. Triangular windows are asymmetric, which enables a user, such as a surgeon, to readily identify the correct orientation of the device that includes such a triangular window alignment aid (for example, ‘triangles point up’ indicating which is the superior surface of the device). Further, when produced using additive manufacturing, triangular windows are self-supporting for the majority of print orientations of the device, which enables triangular windows that define an aperture through a portion of a medical device to be manufactured as an integral part of a device without requiring additional support of structural members that would otherwise have to be removed in a post-manufacturing step.

Further, alignment aids in the form of windows, rather than raised indicators, represent subtractive design. Designing a medical device with a window alignment aid results in the device having less material, meaning less foreign material will be inserted into a patient. The window also provides greater surface area for bone on-growth, allows movement of biological factors through the aperture, and provides better viewing of underlying tissue and bone in post-operative scans.

Integrally formed devices, for example with integrated plates, reduce the number of devices and their associated insertion kits needed in the operating room. A reduced number of items in an operating room reduces time to sterilise and arrange items for the procedure and avoids clutter and confusion that might arise when many devices and associated insertion kits are present. In some embodiments, the integrated plates are patient-specific plates.

Integrally formed devices that are customised based on a patient's anatomy provide a contoured ‘lock-in-key’ fit of the patient-specific devices with the adjacent anatomy, thus reducing the time taken for implantation, the need to remove (good) endplate bone from the vertebral bodies to accommodate the device and difficulties around intra-operative positioning of the device (as the patient-specific devices auto-locate).

The use of additive manufacturing, also known as 3D printing, enables the rapid production of integrally formed medical devices that are customised for specific patients. Rapid production enables customised devices to be used for tumour and trauma patients indicated for surgery, where there is typically a pressing time constraint with the surgeries needing to occur very soon after the initial diagnosis by the surgeon. Conventional processes do not typically allow customised devices to be produced in time for such surgeries.

The embodiments disclosed herein incorporate 3D printable design features suitable for patient-specific interbody devices. Such features include, for example, but are not limited to: integral screw fixation; locking cap/anti-backout features; insertion instrumentation interfaces; radiographic alignment assessment features; anti-expulsion teeth; device identifiers. Device identifiers may be, for example, a string of numbers and/or characters uniquely assigned to each medical device.

When generic ‘off-the-shelf’ spinal devices do not fit with the adjacent anatomy, the anatomy is typically surgically altered to accommodate the device. In the case of spinal interbody and corpectomy devices, this can involve removal, by surgical resection, of ‘good’ (stiffer modulus of elasticity [E]˜12-22 GPa, stronger) endplate bone. Removal of endplate bone means that devices are partially seated on the underlying weaker cancellous bone, which is less stiff (E=˜100-500 MPa). The softer cancellous bone provides a less strong foundation for the device seating, meaning that the device can subside into the vertebral body over time. Subsidence reduces the interbody height between the vertebral bodies adjacent to the device, which reduces the height of the neuroforaminal apertures, in turn reducing the decompression of the neural structures initially achieved by the intervention (i.e., the insertion of the spinal device).

Alternatively, if the endplate bone is not removed and a generic device is implanted, there will typically be only a few points of contact (minimal contact surface area) between the device and the anatomy, due to mismatch in the shape of the device and adjacent anatomy. Low contact surface area between device and anatomy means high pressure, or stress, at the contact points, sometimes termed ‘stress hot-spots’. Such stress hot-spots can also result in subsidence, as the endplate bone is unable to withstand the concentrated loads in these areas.

A good fit between the patient-specific devices and the (interbody) space into which they are implanted means that the devices often ‘auto-locate’ into the planned position. That is, features of the patient-specific devices interact with the contacting anatomy so as to achieve a best fit. In some embodiments that utilise alignment aids, the final depth and orientation positioning of the implanted devices can be assessed using the alignment aids, such as alignment apertures/windows, for example, in combination with intra-operative fluoroscopy.

In some embodiments, it is advantageous to combine the methods for plate customisation and interbody device customisation disclosed in AU 2016900216 and PCT/AU2017/050056, such as to form medical devices that are sized and/or contoured for a particular patient. For example, Anterior Lumbar Interbody Fusion (ALIF), Anterior Cervical Discectomy and Fusion (ACDF), Lateral Interbody Fusion (XLIF/LLIF/OLIF) devices or corpectomy devices optionally feature a plate warped to fit a patient's anatomy that is an integral part of, rather than secondarily attached to, a patient specific device. In one embodiment, this attachment is through manufacture as a 1-piece, integrally formed item. In other embodiments, a plate is attached to a body region of a medical device using screws or other fastening devices, or using frictional contact.

In these medical devices that include both a plate and a body region of a medical device, the patient specific plate can act as an insertion depth ‘stop’ mechanism, meaning that the device is inserted until the plate contacts the vertebral body. The plate can also increase options for integral screw positioning and trajectory, allowing easier intra-operative screw insertion and/or certain regions of vertebral body bone to be reached or avoided.

In another embodiment, a warped plate integrated on the superior and/or inferior device-anatomy surface is used to increase the device foot print, thereby increasing the device contact surface area, spreading the load evenly across the adjacent anatomy (bone), and reducing the potential occurrence of stress hot-spots leading to subsidence.

In another embodiment, integral screw fixation holes in the device are threaded so as to cause a friction (‘cold weld’) lock with a threaded fixation screw head.

### Anterior Cervical Discectomy and Fusion (ACDF) Devices

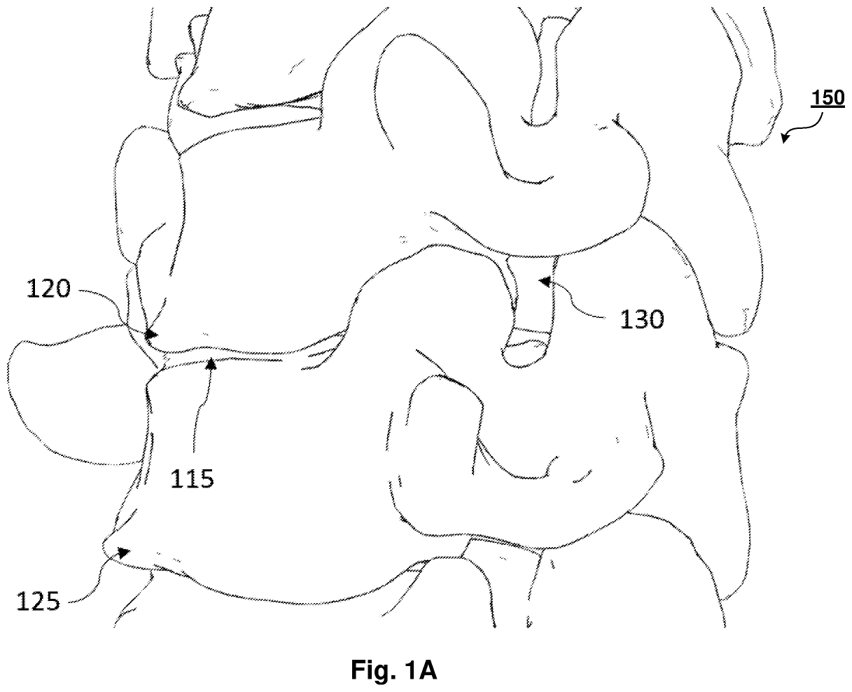

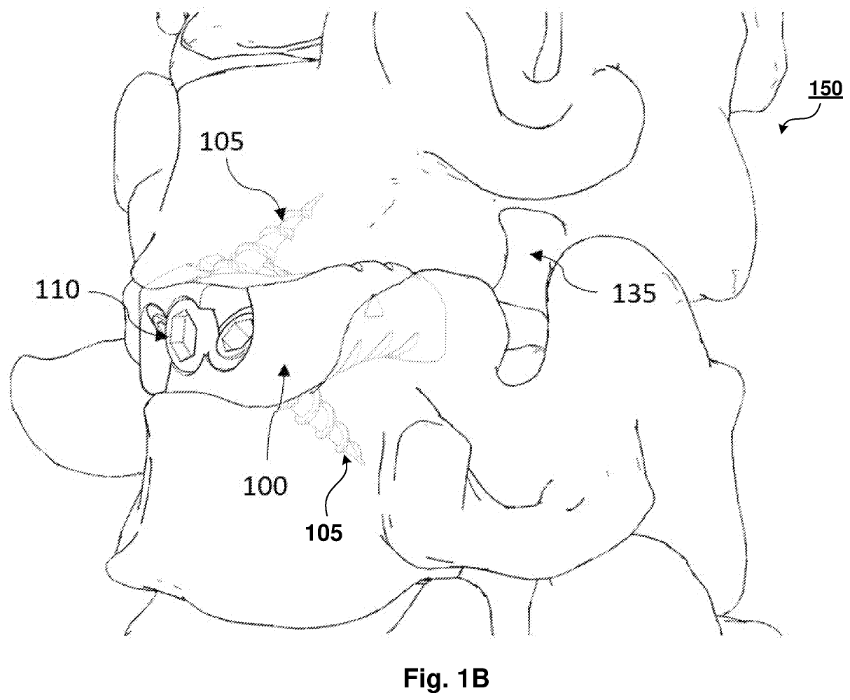

FIGS. 1A and 1B relate to the use of a spinal device to restore the interbody and neuroforaminal height in a patient having a collapsed/pathological cervical (neck) spine intervertebral disc. FIG. 1A illustrates a pre-operative pathological cervical spine level, being a portion of a human spine 150 that includes a superior vertebral body 120 and an inferior vertebral body 115, separated by a collapsed/pathological disc 115. As the disc 115 has collapsed or is otherwise compromised, the interbody spacing between the superior vertebral body 120 and the inferior vertebral body 125 and neuroforaminal spacing 130 have been reduced.

FIG. 1B illustrates the portion of the human spine 150, after surgical intervention using a spinal device in the form of an anterior cervical discectomy and fusion (ACDF) device 100. The ACDF device 100 has been inserted between the superior vertebral body 120 and the inferior vertebral body 125 to restore the interbody and neuroforaminal height 135.

In the example of FIG. 1B, the ACDF 100 is a spinal device that has been customised to fit the anatomy of the patient. In particular, the upper and lower surfaces of the ACDF 100 have been contoured to fit well with the anatomy of the patient. In some implementations, the anatomy of the patient refers to a generic intervertebral spacing, in which case the ACDF 100 is contoured to fit well in that space between two vertebrae. In alternative implementations, the anatomy of the patient refers to the particular anatomy of the individual patient, in which case the ACDF 100 is a patient-specific model contoured to fit well with that particular patient. Contouring of the surfaces may be achieved using one of the methods of AU 2016900216 and PCT/AU2017/050056, parametric CAD methods based in Bezier surfaces, Non-uniform rational B-spline surfaces, Catmull-Clark (or other styles) of subdivision surfaces, or other suitable contouring or shape warping methods, such as those described in Australian Patent Application No. 2021902738, filed 25 Aug. 2021 in the name of 3DMorphic Pty Ltd, the entire content of which is incorporated by reference as if fully set forth herein.

The ACDF 100 in this example utilises integral screw fixation to secure the ACDF 100 to the superior vertebral body 120 and the inferior vertebral body 125. The screw fixation is implemented by a pair of screws 105 that are received through first and second screw apertures in the ACDF 100. The ACDF 100 also includes a screw locking cap 110. The screws 105 combined with the fit of the ACDF 100 with the anatomy creates immediate stabilisation of the instrumented level, giving suitable biomechanical conditions for bone growth and interbody fusion to occur. The locking cap 110 prevents the screws 105, once locked in position, from backing out.

In some embodiments, the angles of the first and second screw apertures in the ACDF 100 are designed and manufactured to be offset relative to each other at a predetermined angle, such that when the pair of screws 105 are screwed into the superior vertebral body 120 and the inferior vertebral body 125, the pair of screws 105 act to pull the vertebral bodies 120, 125 together in a predetermined manner. The offset angle may be adjusted based on user preference, surgical access, and the amount of leverage required to be applied to each of the vertebral bodies 120, 125.

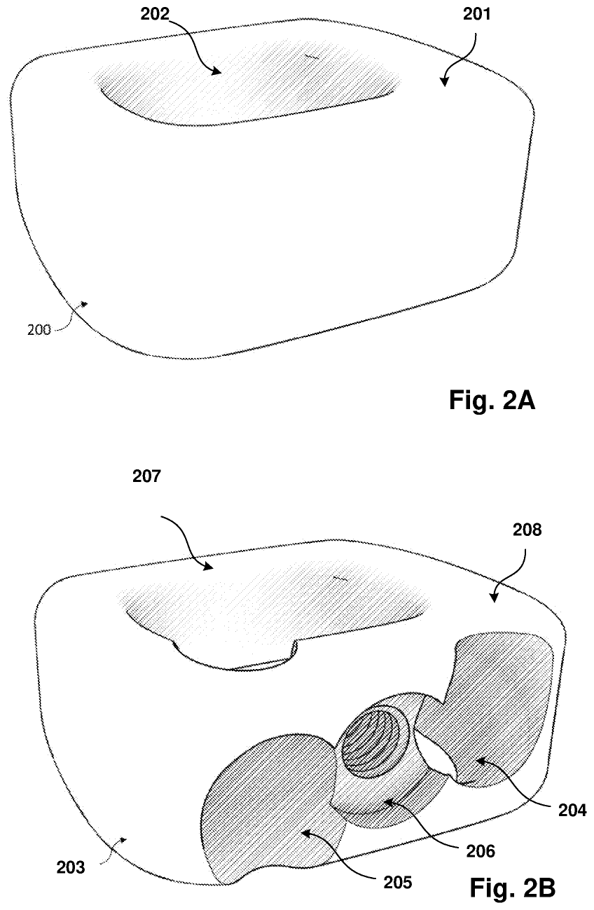

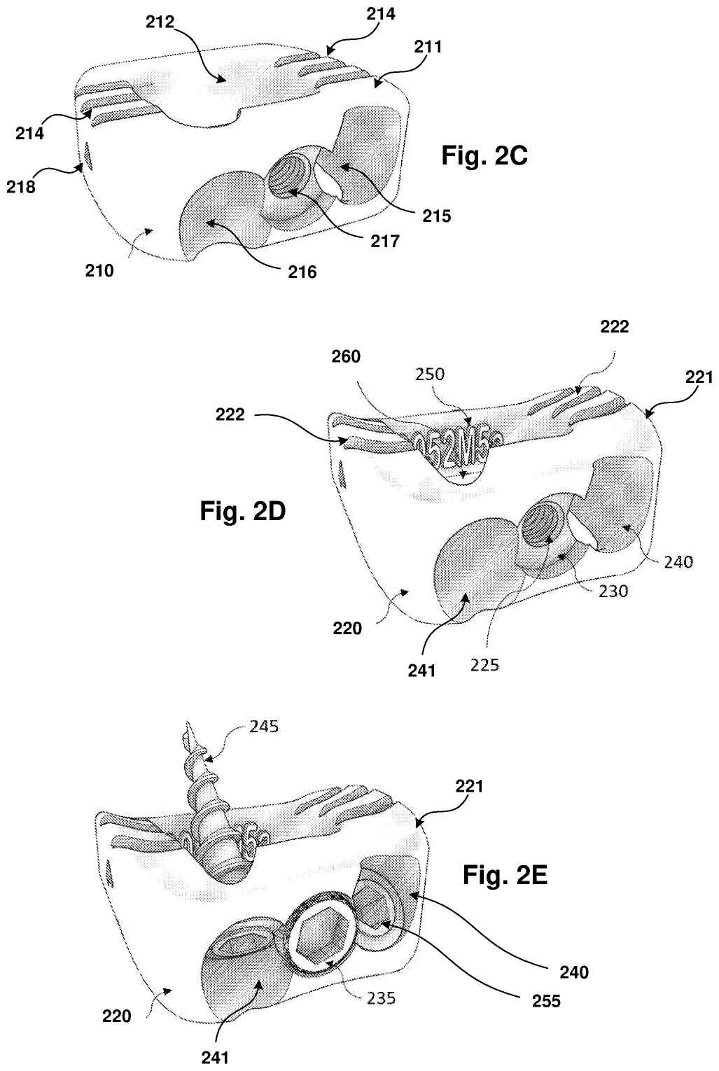

FIGS. 2A to 2C illustrate various ACDF devices. FIG. 2A shows a generic ACDF device 200. The generic ACDF device 200 is generally the shape of a trapezoidal prism having a body portion 201 that is roughly shaped for positioning between adjacent vertebrae. The generic ACDF device 200 also includes a graft window 202 defining a vertical aperture through the body portion 201, in which graft material, such as autograft, allograft, or synthetic bone graft substitutes, can be packed. The presence of the graft window 202 results in the device 200 having an external surface and an interior surface around the defined aperture.

FIG. 2B shows a generic ACDF device 203 having a body portion 208 and a graft window 207 defining a vertical aperture through the body portion 208. The device 203 also has two angled holes 204, 205 for receiving integral screws to secure the ACDF device 203 to the adjacent vertebrae. The device 203 also includes a threaded hole locking cap and/or insertion instrumentation interface 206 recessed into a front (anterior) surface of the device 203. The threaded hole interface 206 is configured to receive a locking cap (not shown) that when screwed into position in the threaded hole is flush with the anterior surface of the device 203 and prevents screws inserted into the holes 204, 205 from backing out.

The generic devices 200, 203 are symmetrical and include geometric shapes. Consequently, the upper and lower surfaces of the devices 200, 203 will not mate very well with the surfaces of the vertebrae between which the devices 200, 203 are inserted. As noted above, points of contact between the vertebrae and the devices 200, 203 may cause stress hot-spots and/or subsidence as the load is concentrated in a relatively small surface area. Further, gaps between the vertebrae and the generic devices 200, 203 will hinder the growth of both on and into the generic devices 200, 203.

FIG. 2C is an ACDF device 210 having a body portion 211 and a graft window 212 defining a vertical aperture through the body portion 211. The device 210 also has two angled holes 215, 216 for receiving integral screws to secure the ACDF device 210 to the adjacent vertebrae. The device 210 also includes a threaded hole locking cap and/or insertion instrumentation interface 217 recessed into a front (anterior) surface of the device 210. The threaded hole interface 217 is configured to receive a locking cap (not shown) that when screwed into position in the threaded hole is flush with the anterior surface of the device 210 and prevents screws inserted into the holes 215, 216 from backing out.

The ACDF device 210 also includes an alignment aid 218 located on a side surface of the device 210. In the example of FIG. 2C, the alignment aid 218 is a triangle window that projects between an exterior surface of the body 211 to an interior surface of the graft window 212. The triangle window 218 is asymmetric, enabling a surgeon to readily identify the correct orientation of the device 210. In some embodiments, multiple alignment aids are provided around the device. In one embodiment, a pair of alignment aids 218 are provided on opposing sides of the device 210.

The ACDF device 210 also optionally includes one or more sets of anti-expulsion teeth 214. In the example of FIG. 2C, two sets of teeth 214 are provided on opposing sides of the device 210. The anti-expulsion teeth 214 are presented as one or more elongated depressions in the upper surface of the device 210 and provide traction between the device and adjacent tissue when implanted. Such anti-expulsion teeth 214 are readily printed using additive manufacturing.

FIGS. 2D and 2E illustrate a patient-specific ACDF device 220 having a body portion 221 that is integrally formed and is shaped for the anatomy of a specific patient. The device 220 includes a graft window 260 defining a vertical aperture through the body portion 221 and in which graft material, such as autograft, allograft, or synthetic bone graft substitutes, can be packed. The device 220 also includes a pair of angled holes 240, 241 for receiving fixation screws to secure the device 220 to adjacent vertebrae.

The device 220 also includes opposing sets of anti-expulsion teeth 222 that match the contours of adjacent anatomical structures, being vertebral endplates of the superior vertebra to which the device 220 is to be attached. In some embodiments, the surface of the body region 221 on which the anti-expulsion teeth 222 are located is warped to match the anatomy of a patient. Such warping may be performed, for example, one or more of the methods of the applications AU 2016900216, AU 2021902738, and PCT/AU2017/050056, or other suitable methods.

The device 220 further includes a threaded hole 225 is recessed 230 into a front (anterior) surface of the device 220 so that, a locking cap (not shown) when screwed into position in the threaded hole is flush with the anterior surface of the device 220. The threaded hole 225 may be used to attach a medical device insertion instrument to the ACDF device 220. The recess 230 around the threaded hole 225 increases the stability of the attachment of the insertion instrument to the ACDF device 220. Medical device insertion instruments typically use a threaded rod or handle that are adapted to be attached to the threaded hole 205 to allow the surgeon to hold and manoeuvre the device 220.

In one embodiment, the threaded hole 225 is formed during an additive manufacturing process, such as 3D printing. In another embodiment, the threaded hole 225 is machined after the rest of the device 220 has been manufactured, such as by 3D printing or other mode of manufacture.

FIG. 2E shows the ACDF device 220 of FIG. 2B with a first screw 245 inserted through hole 241 and a second screw inserted through hole 240. FIG. 2C also shows a locking cap 235 inserted into the threaded hole 225.

In the examples of FIGS. 2D and 2E, the device 220 has an identifier 250 that can be utilised to identify the device 220. The identifier 250 may be a model number, serial number, or a Unique Identifier (sometimes referred to as a Unique Device Identifier, or UDI). The identifier may also be/contain one or a combination of: a manufacturing batch/lot number; a device/product code; information on patient/health care provider; information on device size; and an aid determining which way is ‘up’ and which way is ‘down’ for an asymmetric device.

In the example of FIGS. 2D and 2E, the identifier 250 is raised or embossed on an internal surface of the device 220, such that the identifier 250 stands proud of the internal surface of the graft window 260. In alternative embodiments, the identifier 250 is debossed or formed as an indentation in a surface of the device 220. In some applications, a raised identifier is preferred, as an indented identifier may provide a potential structural weakness in the device 220. Further, a raised identifier provides increased surface area for bone cell attachment and growth. When the identifier is located in the graft window 260 as a raised projection from the inner surface, the identifier 250 also assists in holding the graft in the graft window 260. A raised identifier is also easier to remove manufacturing debris (e.g., for 3D printing, powder beads) from compared to a debossed identifier.

In some embodiments, the identifier 250 is 3D printed on the device 220 during manufacture, which prevents any mixing up of devices during the manufacturing processes, such as between devices intended for different patients or devices directed to different spinal levels within the same patient.

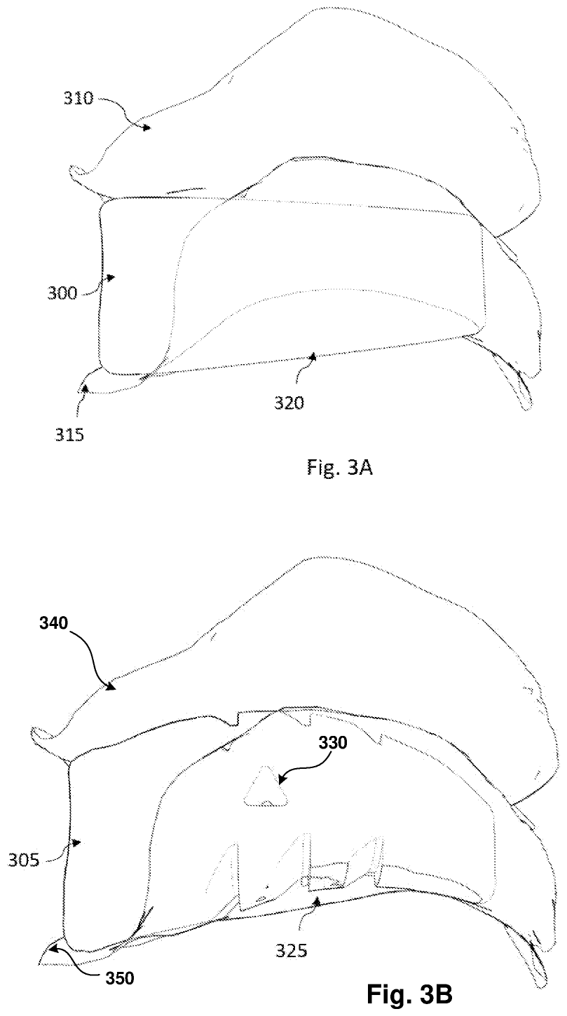

FIGS. 3A and 3B provide a comparison between the fit of a generic ACDF device 300 and that of an ACDF device 305 contoured to fit a patient anatomy, when surgically inserted between adjacent vertebrae.

FIG. 3A shows the generic device 300 positioned in an interbody space defined by adjacent superior 310 and inferior 315 vertebral endplates. FIG. 3B shows a device 305, contoured to fit a patient anatomy, positioned in an interbody space defined by adjacent superior 340 and inferior 350 vertebral endplates. The device 305 has been warped to fit the anatomy of the patient. An inferior region 320 of the generic device 300 protrudes through the inferior endplate 315. In contrast, the corresponding inferior region 325 of the patient-specific device 305 fits well with the inferior endplate and does not protrude. Depending on the implementation, the device 305 may be a patient-specific device warped to fit the anatomy of that individual patient. In alternative implementations, the device 305 may be warped to fit a more generic patient anatomy, such as defined by a particular demographic or other parameters.

In the embodiment of FIG. 3B, the device 305 includes an alignment aid in the form of a triangular window 330 forming an aperture through the side walls of the device 305 is shown. In this example, the triangle points up towards the superior/cranial vertebral body and allows for instant intraoperative assessment of the device orientation during implantation. This is particularly important for devices such as device 305 that are asymmetrical and designed to fit in one orientation only.

The alignment aids on devices also act as radiographic alignment and positioning assessment features. Printing a radiographic alignment assessment window 330 into the device prevents the need to insert marker beads, or other radiographic alignment or positioning features, into the device bodies after initial manufacture, which is currently common practice. Avoiding adding additional markers reduces: the chances of these markers coming loose from the device after implantation; the chances of corrosion interactions occurring between different materials; and radiographic artefacts caused by the markers, which are typically highly radio-dense materials.

The apertures are subtractive design radiographic alignment assessment features. These differ from additive processes (e.g., post [initial manufacture] addition of marker beads) typically used to include radiographic alignment assessment features in devices as they occur at the design, rather than during the physical manufacturing stage. The apertures also differ compared to traditional radio-dense marker beads in that the apertures create a feature with lower (rather than higher) radio-density to allow for alignment assessment.

The apertures are designed to be manufacturable by 3D printing without the need for additional printing support for the surfaces of the apertures, which increases manufacturing efficiency by reducing the need for post-printing removal of support. Reducing printing support also reduces the risk of un-removed supports remaining on the device with the potential to detach after insertion into the body.

The apertures also increase the surface area for bone to grow onto/into the device, increasing the chance of osseointegration and device-anatomy construct stability and allow for biological elements (cells, factors) to move into and out of the device graft window to aid with the bone fusion process. The apertures are straight, or otherwise aligned, to provide ‘line-of-sight’ post print powder removal possibilities, such as through peening/blast (pressurised gas propelled particulate) methods. The apertures can be used post-operatively during follow-up radiographic examination by assessment of the relative position and angles of the apertures. Changes in relative position and angles of the apertures in imaging taken at different follow-up time points can be used to identify and quantify changes in device alignment occurring through device migration and/or device subsidence.

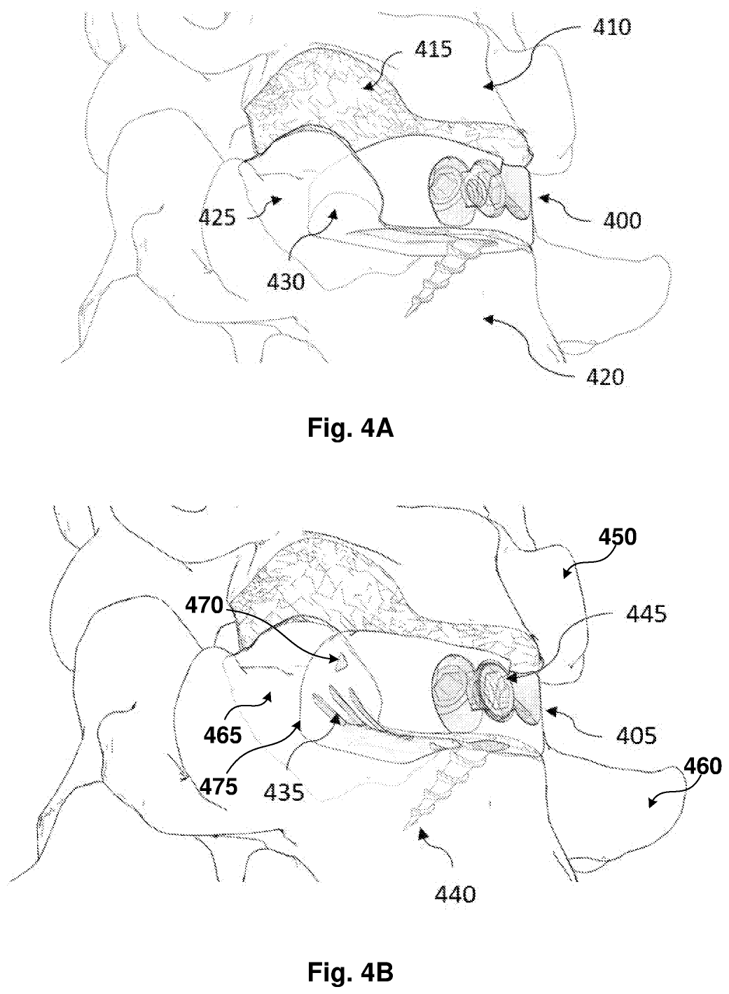

FIGS. 4A and 4B provide a comparison between a generic ACDF device and a better fitting, asymmetric ACDF device. FIG. 4A shows a generic ACDF device 400 positioned in an interbody space between superior 410 and inferior 420 vertebral bodies. The shape of the interbody space is defined by the superior 415 and inferior 425 vertebral endplates. The geometry of the generic device 400 does not fit well in the interbody space, which is shown by an inferior region 430 of the device 400 protruding through the inferior endplate 425.

In contrast, FIG. 4B shows an ACDF device 405 that fits the anatomy of the patient, positioned in an interbody space between superior 450 and inferior 460 vertebral bodies, wherein a corresponding inferior region 475 of the device 405 does not protrude through the inferior endplate 465. The device 405 is shown with an integral screw 440 and locking cap 445 to secure the device 405 in place.

The device 405 shown in FIG. 4B also includes an alignment aid 470 in the form of a triangle window defining an aperture through a side of the device 405. The device 405 further optionally includes anti-expulsion teeth 435 that have been warped with the surface of the device 205 to provide a better anatomical fit.

One method of manufacturing the device 405 involves importing a first digital model of a generic ACDF device, wherein the ACDF device has a substantially trapezoidal body with a graft window projecting vertically through the centre of the body, such that the body portion defines a cage. In this example, the first digital model is a triangulated vertex boundary representation that is suitable for 3D printing.

The method imports a target model, being a digital model of a target shape. In some embodiments, the target shape is associated with the anatomy of the patient, such as a part of the anatomy of a particular patient or a part of a more generalised model of human anatomy. In this example, the target shape is the desired interbody spacing between the superior 410 and inferior 420 vertebral bodies. The target model may be obtained by scanning the superior 410 and inferior 420 vertebral bodies in their initial states to generate an initial target model relating to the compressed interbody space, or through methods disclosed in AU 2021902738. That initial target model is then manipulated to the target shape having the desired interbody spacing.

The method then warps at least one surface of the first digital model of the medical device, based on the target shape, to produce a digital model of the better fitting device. An additive manufacturing machine then uses the digital model of the better fitting device to manufacture the medical device.

In some embodiments, the warping of the surface is achieved by determining a warping interpolation function based on relative positions of a set of source points associated with the first digital model and relative positions of the same points projected to the target shape, and applies the warping interpolation function to all vertices of the medical device to generate a second digital model of said patient-specific device.

In other embodiments, the warping is performed using at least one parametric computer aided design (CAD) methods based in Bezier surfaces, Non-uniform rational B-spline surfaces, or Catmull-Clark subdivision of surfaces.

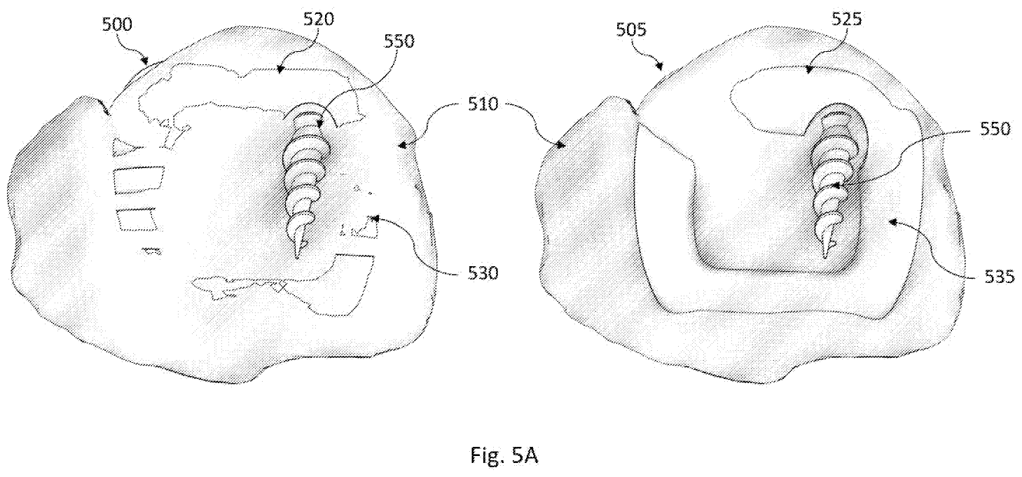

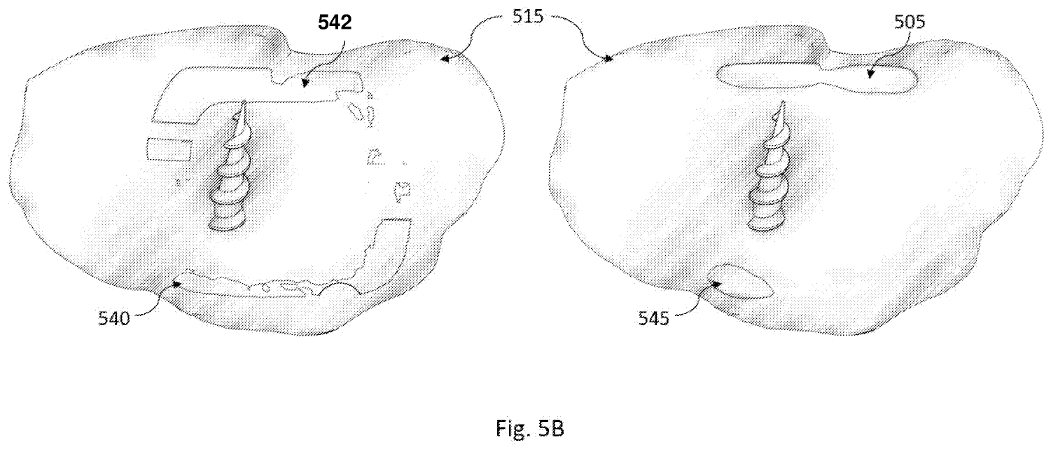

FIGS. 5A and 5B provide a comparison of the contact surface area and penetration of devices through the vertebral body endplates between a better fitting Anterior Cervical Discectomy and Fusion (ACDF) device 500 based on a patient anatomy and a generic ‘off-the-shelf’ type ACDF device 505.

FIG. 5A shows an inferior endplate 510 comparison that illustrates that the device 500 contacts the endplate 510 without penetrating through the endplate 510. The device 500 is secured to the endplate using a fixation screw 550. Good contact between an anterior part 520 of the device 500 and the inferior endplate 510 can be compared with poor contact of an anterior part 525 of the generic device 505, which is similarly secured to the endplate 510 by way of a fixation screw 550. Penetration of the generic device 505 through the inferior endplate 510 is particularly apparent in lateral regions 535 of the endplate 510 compared with no penetration in the corresponding lateral region 530 of the endplate 510 with the device 500.

FIG. 5B shows that at a superior endplate 515 there is good contact between anterior 540 and posterior 542 areas of the better fitting device 500 compared to the generic device 505, which has poor anterior contact 545 and penetration of the posterior part of the device 505 through the endplate 515.

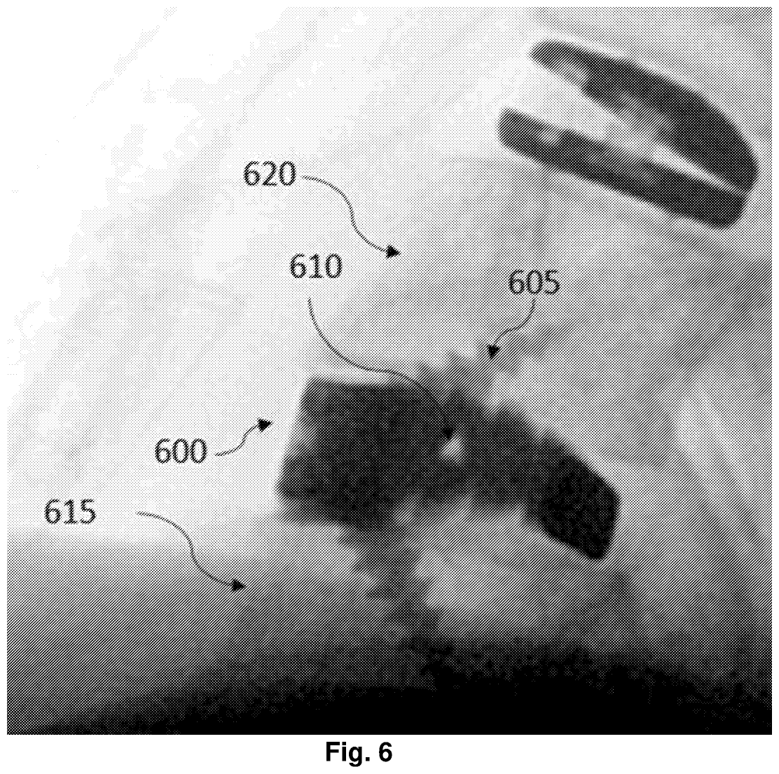

FIG. 6 is an intra-operative x-ray of an ACDF device 600 with integral screws 605 and radiographic alignment feature 610 in the form of triangular apertures. The ACDF device 600 is shown in an interbody space between inferior 615 and superior 620 vertebral bodies.

It is to be noted that the triangle apertures on both the left and right hand sides of the device 600 are aligned with the x-ray machine emitter and receiver, as evidenced by the clear ‘line of sight’ through the triangles. The x-ray is taken in the sagittal (lateral) plane, indicating that the device 600 is aligned in this plane. The triangular windows point up, indicating that the device 600 has been implanted with the correct orientation. The triangular windows act as alignment assessment features in the opposite manner to traditional methods, being high radio-density marker ‘beads’, in that the triangular windows allow alignment assessment through the absence of radio-dense material. This also means that there is no manufacturing stage in which other material is added to the device specifically for the purposes of positioning and alignment assessment.

### Anterior Lumbar Interbody Fusion (ALIF) Devices

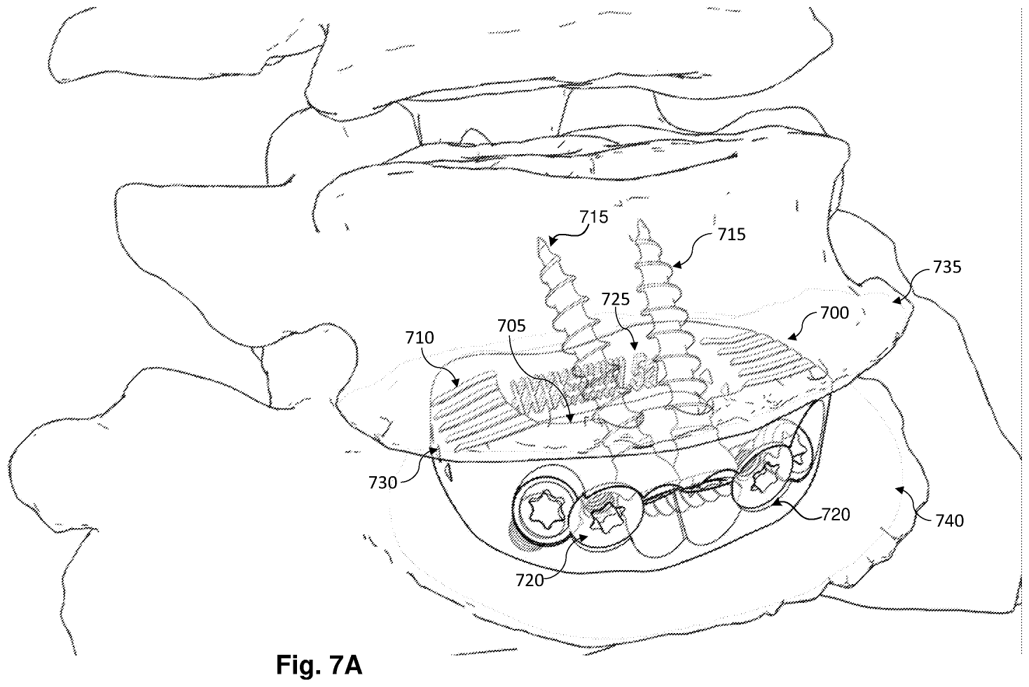

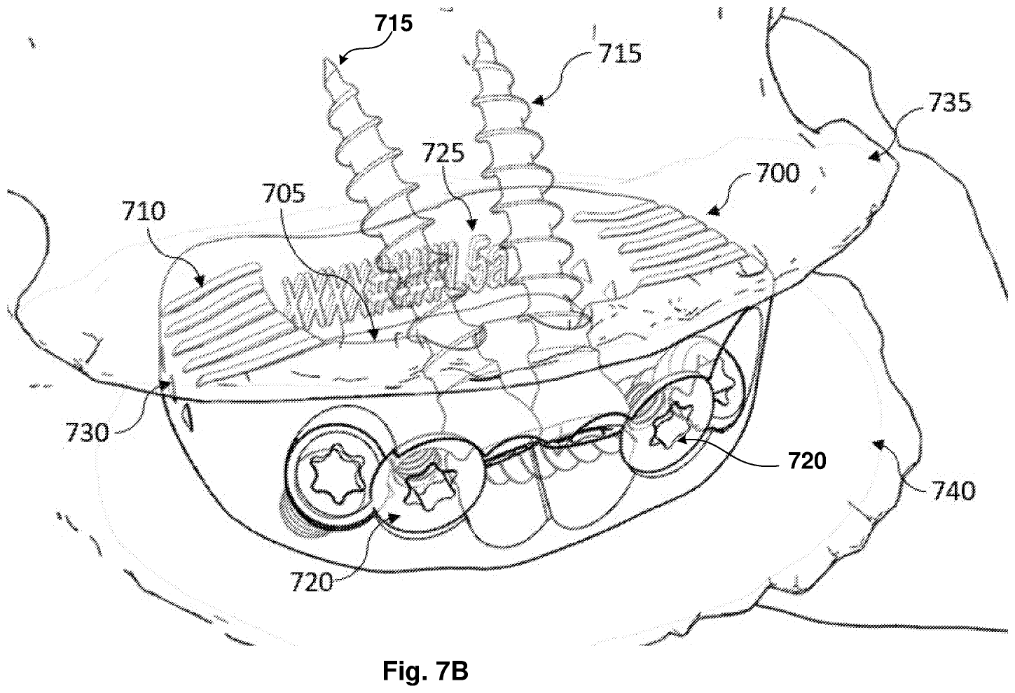

FIG. 7A shows an Anterior Lumbar Interbody Fusion (ALIF) device 700, which in this example is contoured to fit a patient anatomy. The device 700 has a graft window 705 and anatomy contour matching anti-expulsion teeth 710. The device 700 also includes: integral screw fixation 715; screw locking caps 720; embossed (proud) identifier 725 on an interior wall of the graft window 705; and intraoperative orientation assessment and radiographic alignment and positioning assessment triangle apertures 730. The ALIF device 700 is shown in the interbody space between the superior lumbar five (L5) vertebral body 735 and the inferior sacral one (S1) vertebral body 740. FIG. 7B is an enlarged view of the device 700 of FIG. 7A.

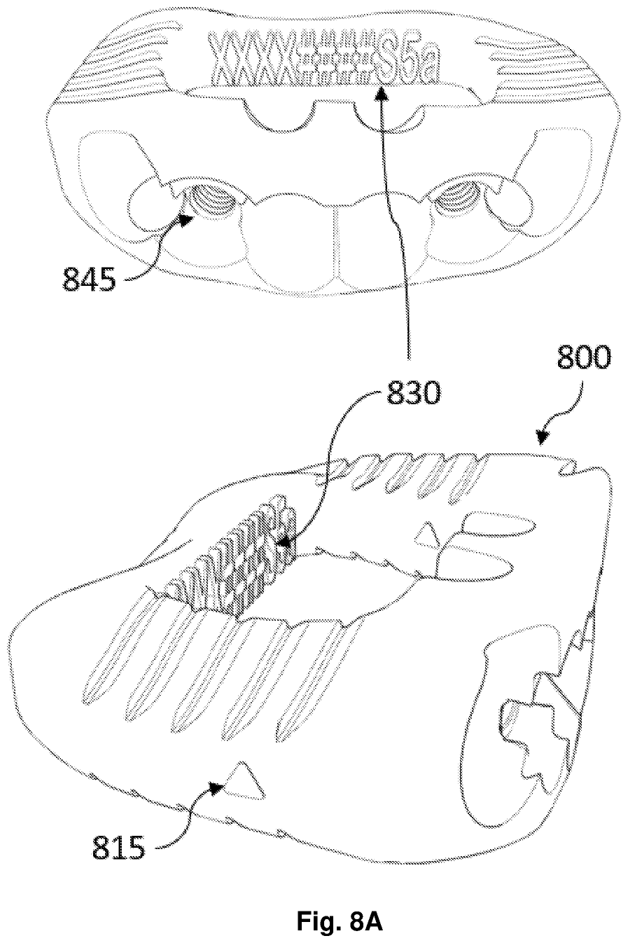

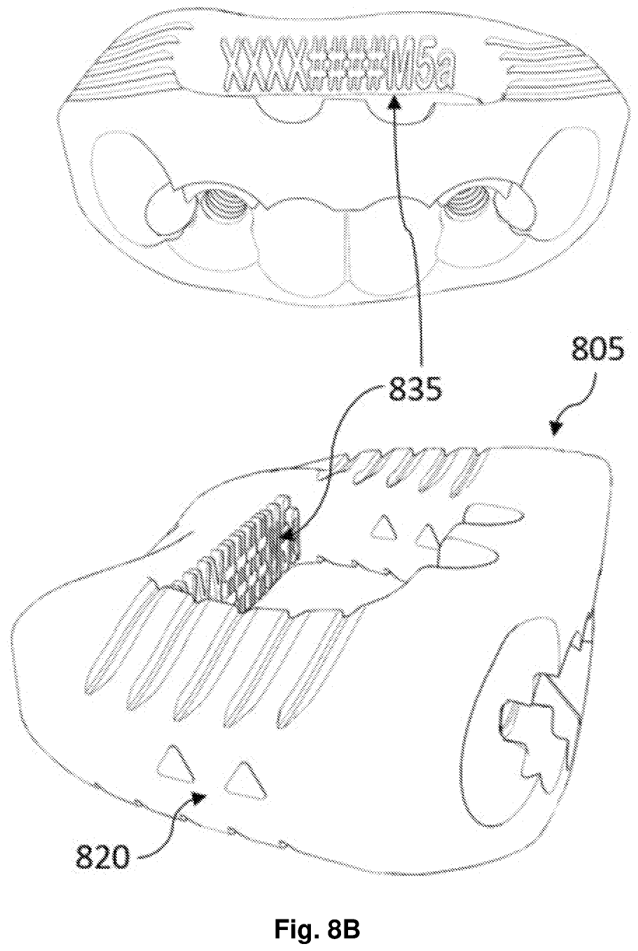

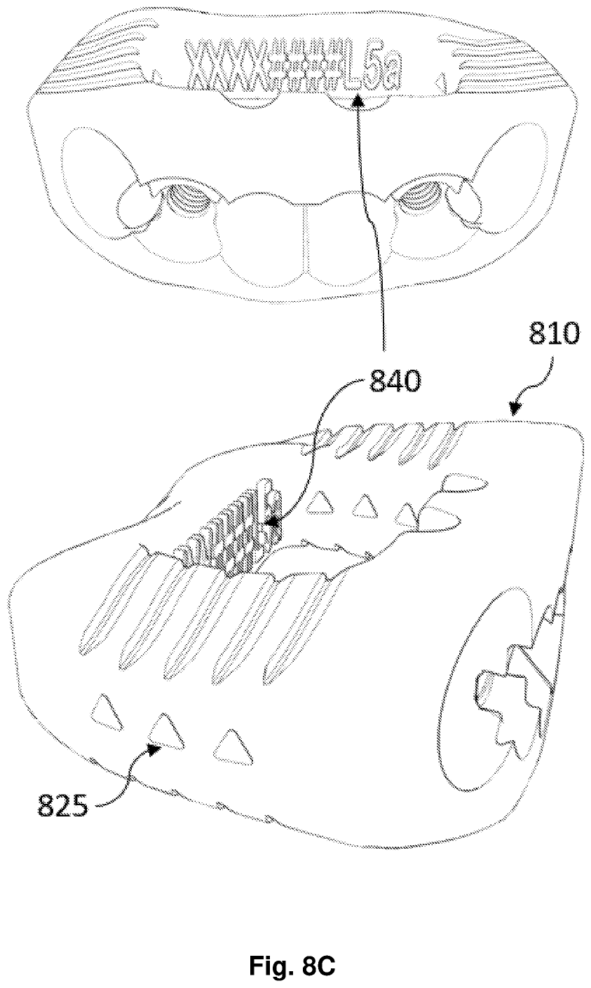

FIGS. 8A to 8C show small 800, medium 805, and large 810 variations of Anterior Lumbar Interbody Fusion (ALIF) devices (the large device is shown in-situ in FIGS. 7A and 7B). The devices 800, 805, 810 differ in respective heights and angles and offer intra-operative options for the surgical team. Typically, the small 800 will be used first as a ‘trial’ to assess the level of distraction and mobility achieved by the surgical discectomy. In the case in which patient-specific devices are being used, providing multiple sizes of patient-specific devices provides the distinct advantage of meaning that no re-usable trial devices are needed in the instrument trays during the surgical procedure, which reduces inventory management costs and infection risks associated with the provision and cleaning of reusable trial devices.

FIG. 8A shows the small device 800 from a side and frontal view, wherein the small device includes a unique identifier 830, a threaded screw hole 845, and an alignment aid 815 in the form of a single triangle aperture.

FIG. 8B shows the medium device 805 from a side and frontal view, wherein the small device includes a unique identifier 835 and an alignment aid 820 in the form of two triangle apertures.

FIG. 8C shows the large device 810 from a side and frontal view, wherein the small device includes a unique identifier 840 and an alignment aid 825 in the form of a three triangle apertures.

The devices 800, 805 and 810 are immediately distinguishable from one another, as the small device 800 has one triangle window 815, the medium device 805 has two triangle windows 820 and the large device 805 has three triangle windows 825. It will be appreciated that other identifying marks may equally be practised to differentiate among different sized devices.

In the examples of FIGS. 8A to 8C, each device 800, 805, 810 has an embossed identifier, wherein the embossed identifier also includes an indication of the size (S for small 830, M for medium 835, L for large 840) as well as a subsequent number, five in this case, which indicates the vertebral level superior to the device (L5).

In the case where a patient is receiving multiple level instrumentation with patient specific devices, the level code allows intra-operative determination of which device is to be used with which interbody level. In this example, the number in the identifier is followed by a letter, which indicates the print run (lot/batch) number of the device, allowing quality traceability of raw materials used in the manufacture of the device.

### Cervical Corpectomy

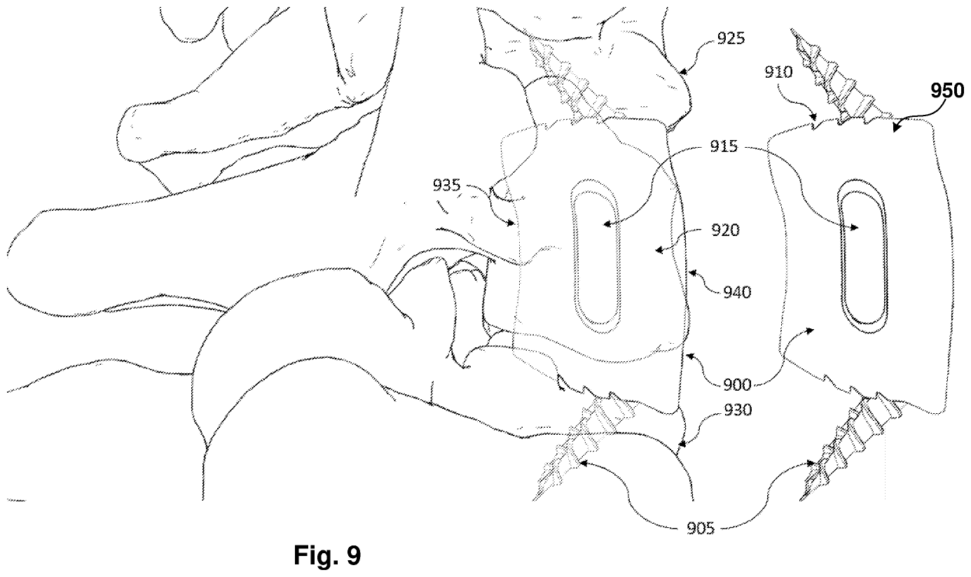

FIG. 9 is a lateral (sagittal plane) view of a cervical corpectomy/vertebrectomy device 900 with integral screw fixation 905, and anti-expulsion teeth 910 contoured to superior 925 and inferior 930 vertebral body endplates. The device 900 is implanted by surgical resection of the midline region of a vertebral body 920.

The cervical corpectomy device 900 features a body 950 through which are defined large at least one lateral aperture/window 915 that: aid graft packing; allow biological material (cells, factors) to move between the vertebral body bone adjacent to the device 920 and the graft contained in the graft window of the device to aid in the bone fusion process; reduce the volume of device material implanted into the body; and allow for intra-and post-operative fluoroscopy alignment assessment. Lateral graft windows also allow for bone growth between graft window and surrounding bone. Such bone growth creates resistance to axial rotation movement/migration of the device by effectively ‘locking’ the device into position. Such bone growth also increases the surface area for axial load sharing between device and surrounding bone, which can reduce the risk of subsidence through reducing endplate bone stress.

A posterior wall 935 of the device 900 is curved away from the spinal canal to move the material of the device away from the spinal cord, allowing for maximum cord decompression and reducing imaging artefact in the vicinity of the cord that the device may cause. An anterior wall of the device is curved to match the natural curvature of the patient's spine.

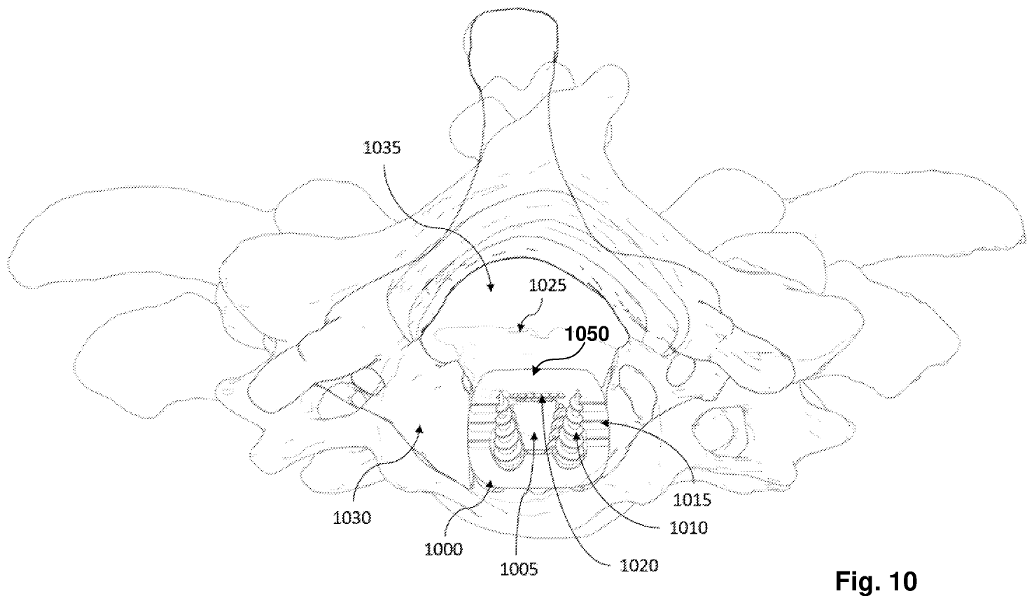

FIG. 10 is a superior (axial plane) view of a cervical corpectomy/vertebrectomy device 1000 having: a body portion 1050; a graft window 1005 defining a central vertical aperture through the body portion 1050; integral screw fixation 1010; anti-expulsion teeth 1015; and an embossed device identifier 1020. Vertebral corpectomy devices can be used to treat vertebral (burst) fractures in which a fracture fragment 1025 is compressing the spinal cord by protruding into the spinal canal 1035. The device 1000 can be implanted from an anterior approach by surgical resection of the midline of the fractured vertebral body 1030, which allows for removal of the protruding fracture fragment 1025 and cord decompression.

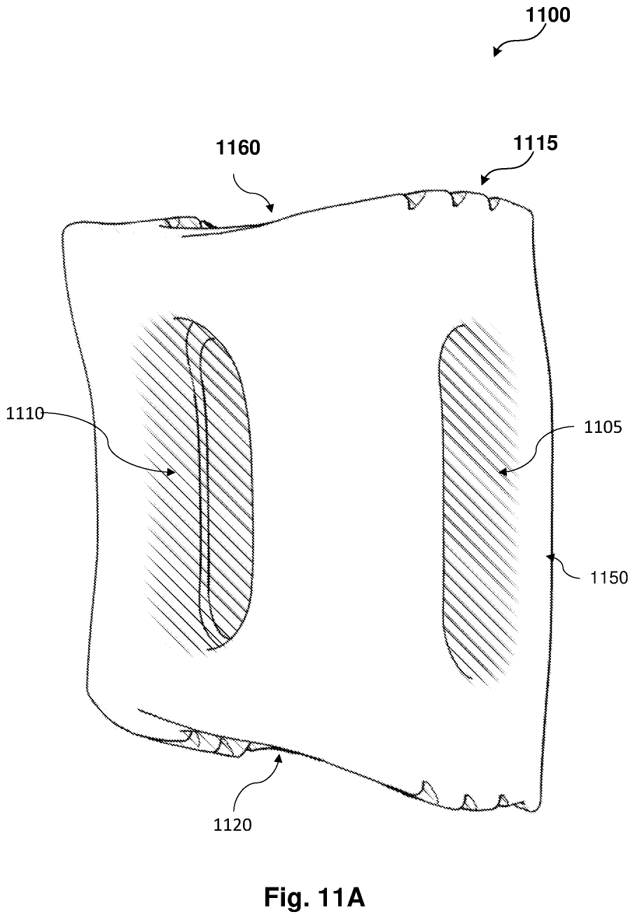

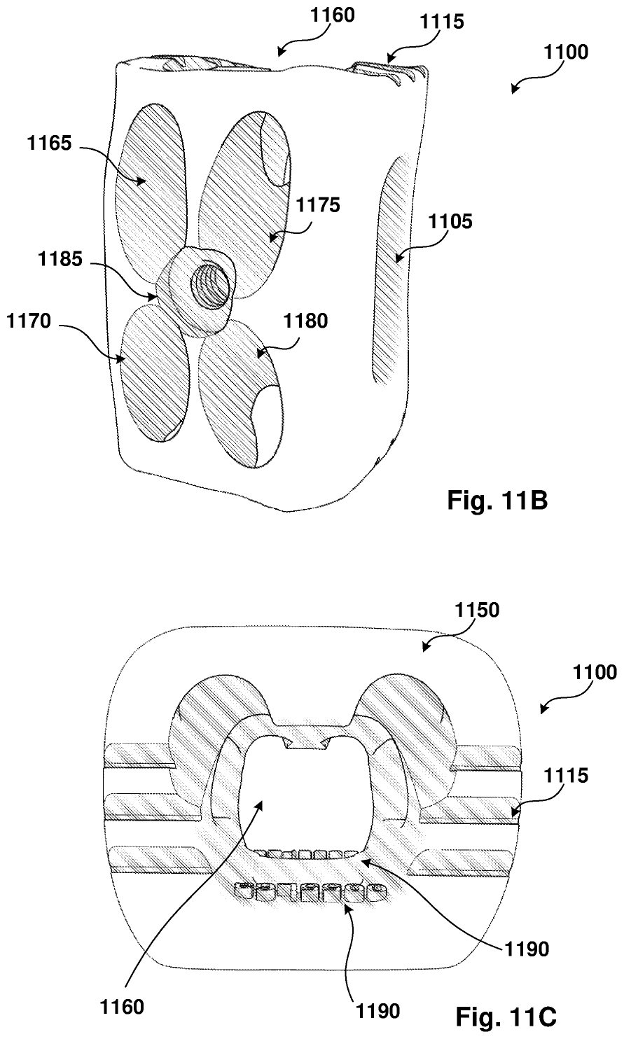

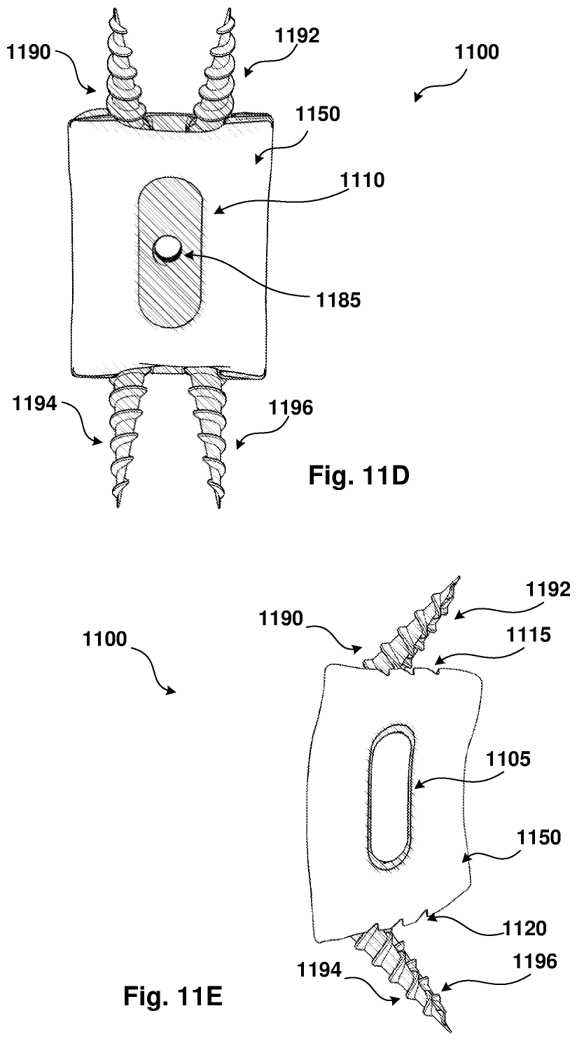

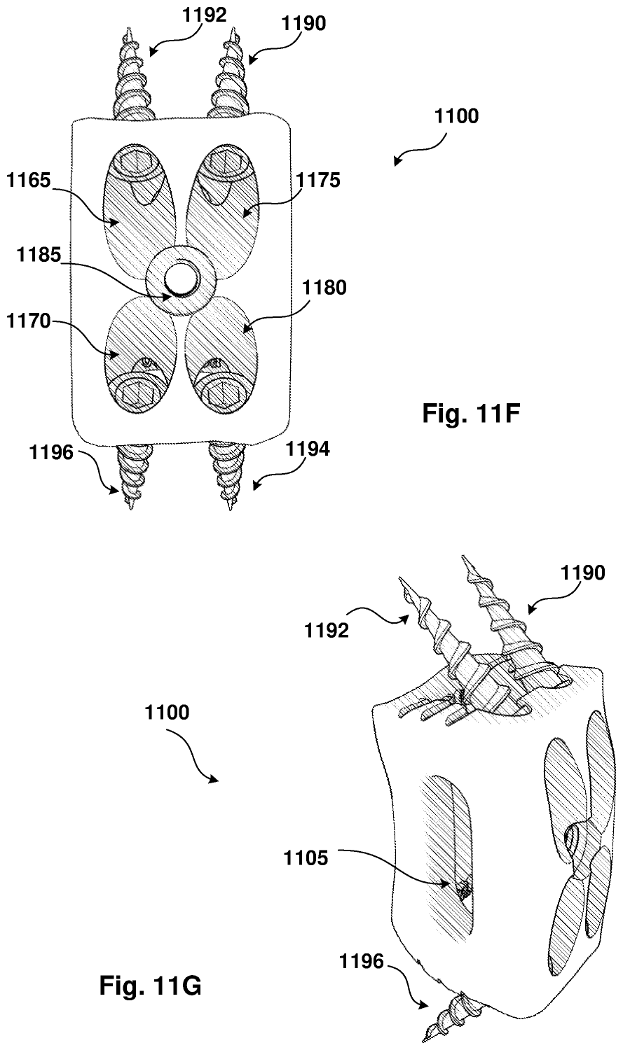

FIG. 11A shows a cervical corpectomy/vertebrectomy device 1100 viewed from a posterior lateral viewpoint. The device 1100 includes a body portion 1150 having: lateral apertures/windows 1105 on opposing faces of the device 1100; a posterior aperture/window 1110; and anti-expulsion teeth contoured to the superior 1115 and inferior 1120 vertebral endplate curves and angles. Note that the superior 1115 and inferior 1120 contours and angles of the device-anatomy interfaces differ from, and are independent to, one another. The device 1100 also has a graft window 1160 defining a vertical aperture through the body portion 1150. The large posterior aperture 1110 aids in graft packing as well as reducing the volume of material in proximity to the spinal cord that could produce device related imaging artefact. For example, in a 3D printed Titanium alloy embodiment of the device 1100, the posterior aperture 1110 aims to reduce the volume of titanium that could cause interference with CT and/or MRI assessment of cord decompression.

FIG. 11B is a front perspective view of the vertebrectomy device 1100 of FIG. 11A. The device 1100 has four screw holes 1165, 1170, 1175, 1180 and a threaded hole which is an insertion instrumentation interface 1185 that is recessed into a front (anterior) surface of the device 1100.

FIG. 11C is a top view of the vertebrectomy device 1100 of FIG. 11A. The device 1100 has identifiers 1190 printed onto the anterior wall of the graft window 1160 at the top and bottom of the device 1100. Identifiers embossed in this way at the top and bottom of the graft window can aid in retention of graft material in the graft window and act to increase the surface area for bone to grow onto.

FIG. 11D is a rear view of the vertebrectomy device 1100 of FIG. 11A, with four screws 1190, 1192, 1194, 1196. The posterior aperture 1110 is viewable in the rear surface of the body region 1150.

FIG. 11E is a side view of the vertebrectomy device 1100 of FIG. 11A, with four screws 1190, 1192, 1194, 1196.

FIG. 11F is a front view of the vertebrectomy device 1100 of FIG. 11A, with four screws 1190, 1192, 1194, 1196 inserted into screw holes 1175, 1165, 1180, 1170, respectively.

FIG. 11G is a left side perspective view of the vertebrectomy device 1100 of FIG. 11A, with four screws 1190, 1192, 1194, 1196 inserted into screw holes 1175, 1165, 1180, 1170, respectively.

### Lumbar Corpectomy Device

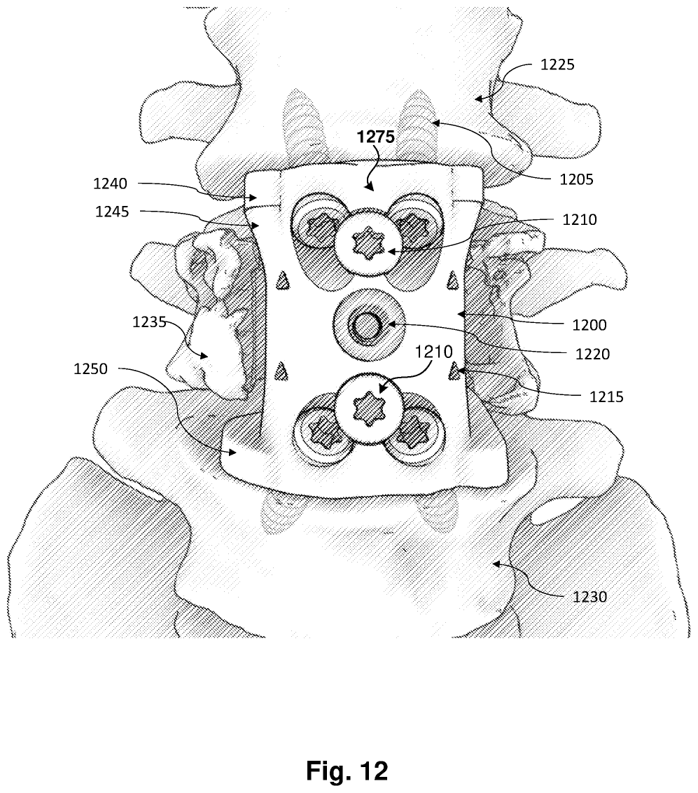

FIG. 12 is an anterior (coronal plane) view of a lumbar corpectomy/vertebrectomy device 1200 having: a body portion; integral screw fixation 1205; locking caps 1210; orientation and radiographic position and alignment assessment triangle apertures/windows 1215; and recessed threaded insertion instrumentation interfaces 1220.

This lumbar corpectomy device 1200 is implanted between the superior vertebral body 1225 and inferior vertebral body 1230 after resection of the midline part of the pathological vertebral body 1235. The device 1200 features integrated warped plates at the superior 1240 and inferior 1250 device-anatomy interfaces. These integrated warped plates increase the contact surface area between device and anatomy, which reduces stress/pressure in the vertebral endplate anatomy, thereby reducing the risk of device migration and/or subsidence.

In some embodiments, warping of the plates is performed using one or more of the methods described in the applications AU 2016900216, AU 2021902738, and PCT/AU2017/050056. In particular, a base version of the device 1200 having regular superior and inferior plates is warped, based on a target shape, being the respective superior and inferior vertebral plates to which the device 1200 is to be attached.

In the example of FIG. 12, the superior aspect integrated plate 1240 is buttressed 1245. This increases the strength of the superior plate 1240, as well as reducing the overhang of the plate, which can increase 3D printing manufacturing efficiency by reducing the support needed to manufacture this part of the device. The integrated warped plates distribute the device-anatomy loads over greater surface area of the anatomy, thereby reducing stress (N/mm2) in the bone and reducing the risk of subsidence of the device into the adjacent vertebral bodies.

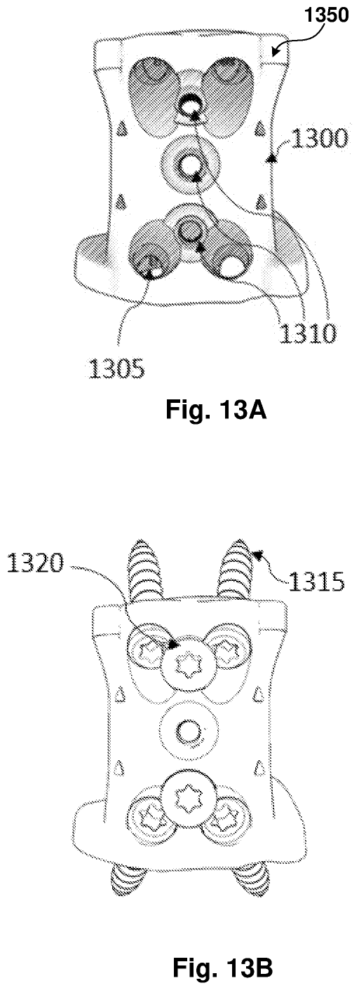

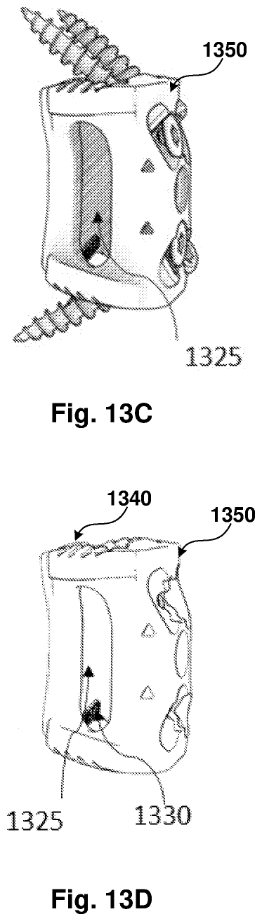

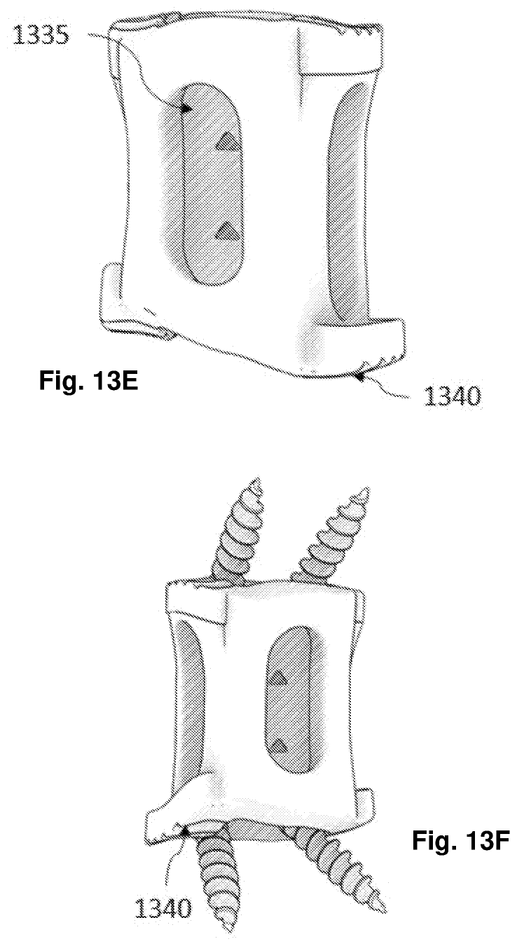

FIGS. 13A to 13F show a lumbar corpectomy/vertebrectomy device 1300 with holes 1305 for integral screws 1315, corresponding to the device 1200 from FIG. 12. FIG. 13A shows the device 1300 having a body portion 1350 enclosing a vertical graft window that defines a central vertical aperture through the device 1300. The body portion 1350 has three threaded insertion instrumentation interfaces 1310, allowing for intra-operative options as to positioning of the insertion instrument and angle of insertion with respect to surrounding tissues. As shown in FIG. 13B, superior and inferior insertion instrumentation threaded interfaces also double as locking nut interfaces 1320, wherein once the device 1300 has been inserted into the patient and secured by the integral screws 1315, locking nuts can be threaded into the insertion instrumentation interfaces 1310 to prevent the screws 1315 from backing out.

FIGS. 13C and 13D show a side perspective view of the device 1300, showing a large aperture 1325 in a lateral wall of the body region 1350. Depending on the implementation, a pair of apertures 1325 is provided on opposing lateral walls of the body region 1350. Alternatively, a single aperture 1325 is provided on one lateral wall of the body region 1350. The aperture(s) 1325 aid graft packing, intra and post-operative radiographic device orientation assessment, reduce the volume of material implanted into the patient, and reduce the potential for imaging artefacts caused by the material of the body region 1350. An embossed identifier 1330 is present at the superior and inferior ends of the internal graft window wall to aid device identification and to hold better the graft packed into the graft window. The warped plate at the superior surface of the body region 1350 optionally includes contoured anti-expulsion teeth 1340.

FIGS. 13E and 13F show posterior perspective views of the device 1300, showing a large aperture 1335 in a posterior wall of the body region 1350. As for the aperture(s) 1325 in lateral walls, the aperture 1335 aids graft packing, intra and post-operative radiographic device orientation assessment, reduces the volume of material implanted into the patient and post-operative radiographic device orientation assessment, reduces the volume of material implanted into the patient, and reduces the potential for imaging artefacts caused by the material of the body region 1350. The warped plate at the inferior surface of the body region 1350 optionally includes contoured anti-expulsion teeth 1340.

### Lateral Approach Devices

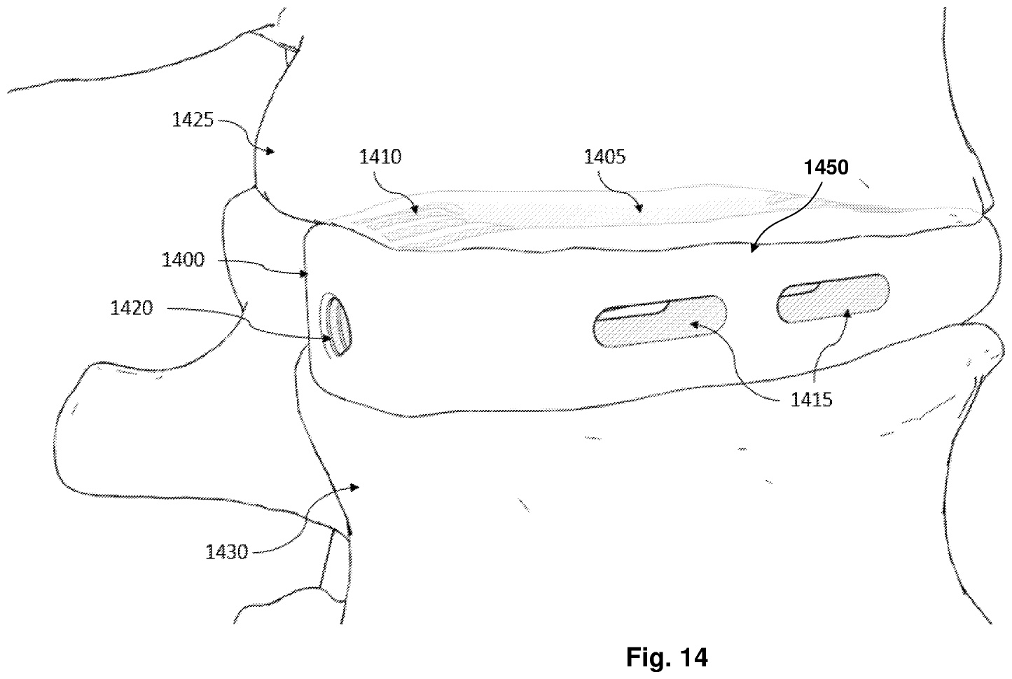

FIG. 14 is an anterior right lateral view of a lateral approach interbody device 1400. Lateral approach devices include: Lateral lumbar Interbody Fusion (LLIF); Oblique Lumbar Interbody Fusion (OLIF); and eXtreme Lateral Interbody Fusion (XLIF). The device 1400 features: a body portion 1450 having a graft window 1405 defining a vertical aperture therethrough; contoured anti-expulsion teeth 1410; apertures/windows 1415 to allow for intra and post-operative radiographic alignment assessment, increase surface area for fusion bone growth, and allow biological elements (cells and factors) to pass into and out of the device graft window; and a threaded insertion instrumentation interface 1420. The device 1400 is implanted between superior 1425 and inferior 1430 lumbar vertebral bodies to treat discogenic pathology and/or spinal alignment pathologies.

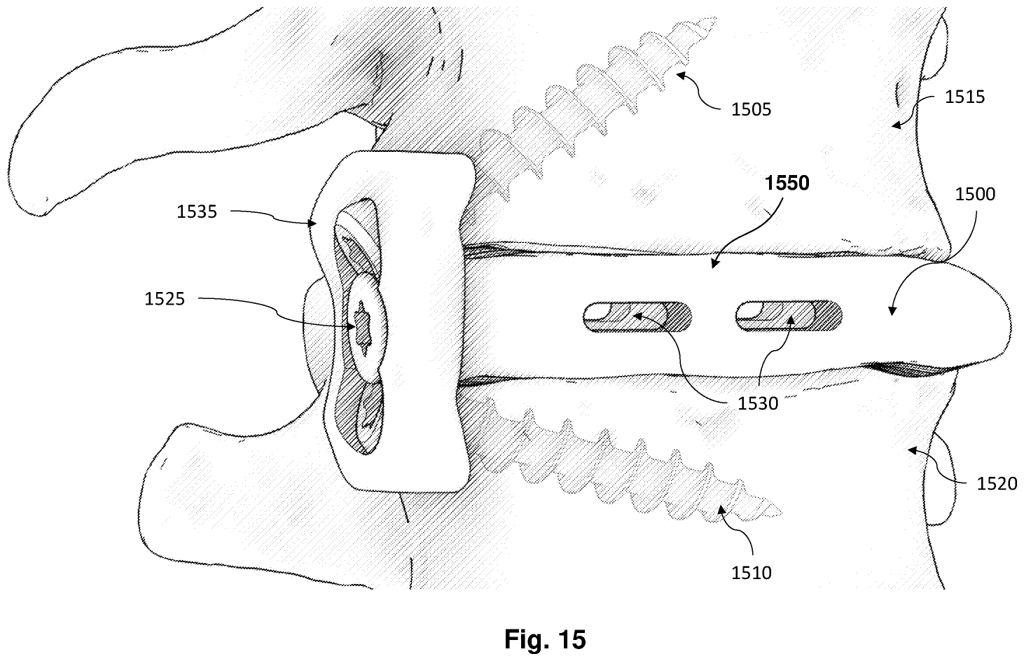

FIG. 15 is an anterior right lateral view of a lateral approach interbody device 1500. The device 1500 includes: a body region 1550; superior 1505 and inferior 1510 integral screw fixation in the superior 1515 and inferior 1520 vertebral bodies; screw locking nut 1525; anterior and posterior wall apertures/windows 1530; an integrated patient-specific warped lateral plate 1535, which: conforms to the patient's vertebral anatomy, thereby being lower profile than a non-conforming plate, which reduces the risk of surrounding soft tissue irritation, and increases contact surface area between device and anatomy to facilitate early bone on-growth (osseointegration) between plate and bone, which increases stability of the construct; acts as a ‘stop’ during implantation; accommodates integral fixation screws for superior and inferior vertebral bodies.

In some embodiments, the integrated warped lateral plate 1535 is warped to match the anatomy by using one or more of the methods of the applications AU 2016900216, AU 2021902738, and PCT/AU2017/050056.

Integral screw fixation in both superior and inferior vertebral bodies is sometimes used if the pathology is treated by lateral interbody device alone, as opposed to a lateral and posterior (rod and pedicle screws) treatment. The trajectories of the screws differ as the screws are designed to achieve different effects. In order to achieve the different effects, the device 1500 may be manufactured with custom angles for the holes to receive the screws 1505, 1510 in order to locate the device 1500 in a desired orientation during surgery.

In general, one or more low (predominantly lateral) angled screws can be used to draw a device further into the interbody space. One or more higher (more vertically) angled screws can create compression between anatomy and device. Combinations of different angled inferior to superior level screws can achieve a combination of effects.

When the shallower angled inferior screw 1510 is inserted first and to the full extent, that inferior screw 1510 draws the device 1500 into the planned depth (of insertion) position, bringing the warped integral plate 1535 into intimate contact with the lateral wall of the vertebral bodies. When the steeper angled superior screw 1505 is inserted second and to the full extent, that superior screw 1505 draws the spine superior to the device 1500 into the planned coronal plane alignment (scoliosis correction) as well as compressing the superior vertebral body 1515 onto the device 1500, which gives the intimate contact between device 1500 and bone necessary for construct stability and bone on-growth onto the device (osseointegration). The realignment of the spine superior to the device 1500 allows for scoliotic (coronal plane) curvature pathologies to be treated. Lordotic (sagittal plane) curvature pathologies are treated by the posterior-anterior ‘wedge’ angle of the interbody part 1550 of the device. Screw angles can be optimised to achieve these effects whilst not blocking the pedicle windows (used for posterior approach pedicle screw and rod constructs).

In some embodiments, medical devices include a set of structural members spanning an aperture defined through a body region. The set of structural members may form a lattice or array of structural members. The structural members may be located across the openings of the aperture, within the aperture, or a combination thereof. Such a set of structural members provides increased surface area for bone on-growth and allows the aperture to be larger in size, thus reducing the overall volume of material in the device. For example, with reference to FIG. 15, one or more of the anterior and posterior wall apertures/windows 1530 optionally includes a set of structural members spanning the respective aperture 1530.

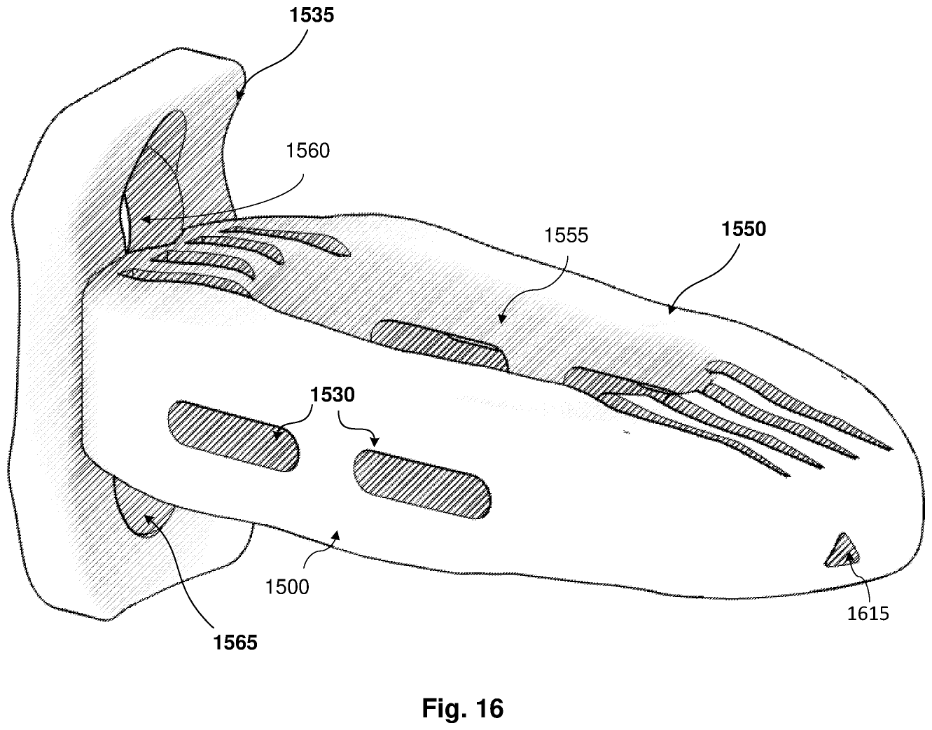

FIG. 16 is an anterior left lateral view of the lateral interbody device 1500 as shown in FIG. 15. The device 1500 features: a body portion 1550 having a graft window 1555 defining a vertical aperture therethrough; a first screw hole 1560 passing through the integrated patient specific lateral plate 1535 and for receiving the superior screw 1505; a second screw hole 1565 passing through the integrated lateral plate 1535 and for receiving the inferior screw 1510; and a triangular aperture/window through a lateral wall, or nose, of the body region 1550 for intra-operative orientation assessment. In some embodiments, the integrated lateral plate 1535 is patient-specific.

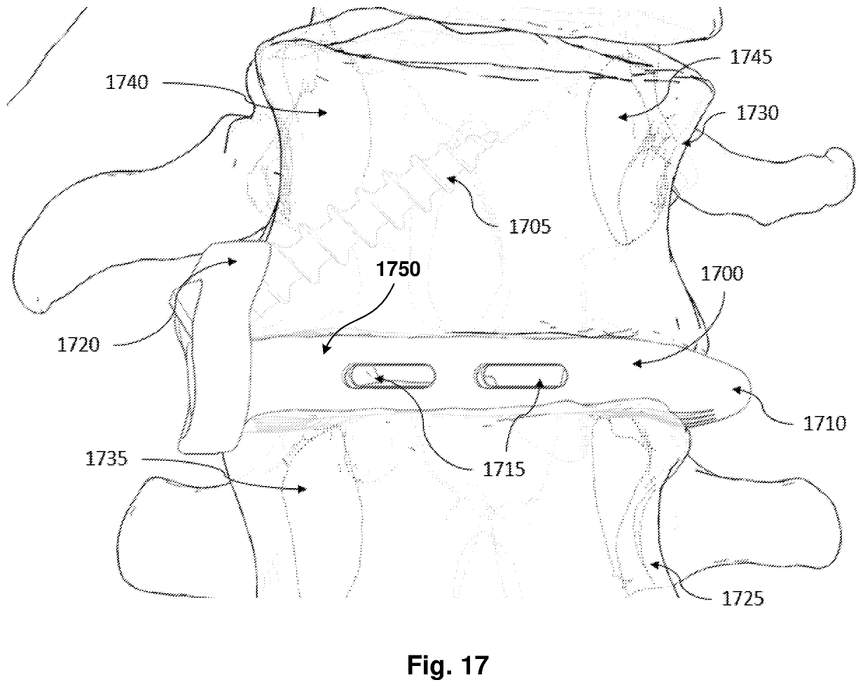

FIG. 17 is an anterior view of a lateral interbody device 1700 with one integral screw fixation screw 1705. The device 1700 features: a body portion 1750 having a smooth, tapered ‘bullet nose’ 1710 to aid device insertions and vertebral body realignment resulting from the insertions; anterior and posterior wall apertures/windows 1715; and an integrated warped patient specific plate 1720 with a screw hole for an integral screw 1705 for the superior vertebral body 1730. The device 1700 is designed to fit in the interbody space between inferior 1725 and superior 1730 vertebral bodies.

The trajectory of the inferior screw 1510 shown in FIG. 15 would cross the ‘pedicle window’ 1735 of the inferior vertebra 1725. Hence, the device 1700, having only a superior screw 1705, is ideal for use in combination with posterior fixation (rods and pedicle screws) as: the integral screw 1705 in the superior vertebral body 1730 avoids the right 1740 and left 1745 pedicle windows; and fixation with only one screw allows for (sometimes more major) coronal plane realignments (scoliosis corrections) to be performed with posterior instrumentation after implantation of the lateral device 1700, which is often the preferred surgical workflow.

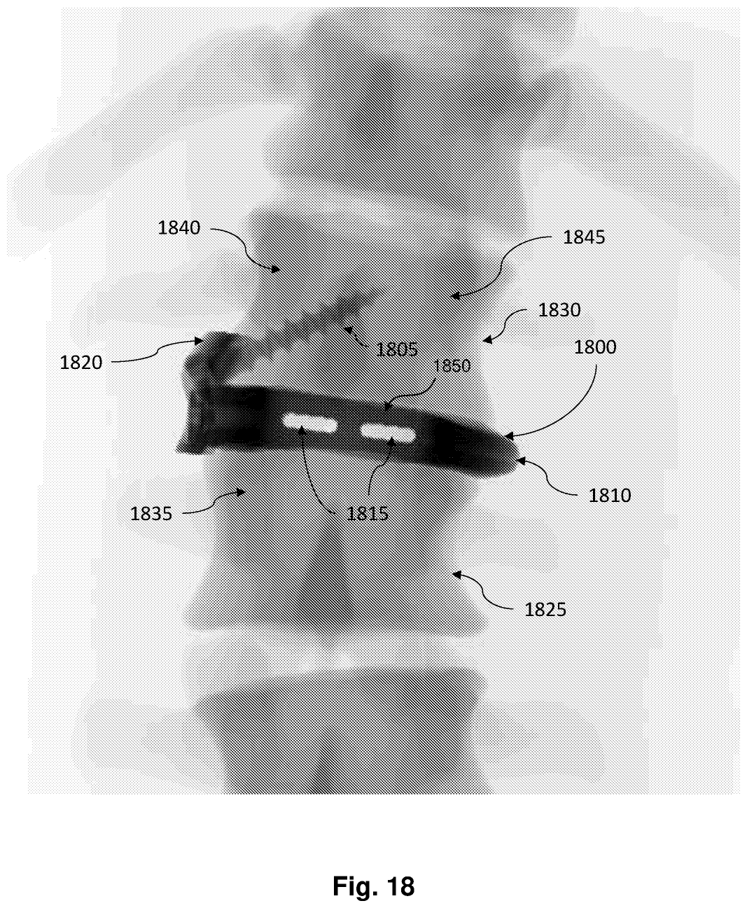

FIG. 18 is a simulated anterior-posterior (AP) x-ray of a lateral interbody device 1800 with one integral screw fixation screw 1805, corresponding to the device 1700 shown in FIG. 17. The integral fixation screw 1805, bullet nose 1810, anterior and posterior wall radiographic alignment assessment apertures/windows 1815, and patient specific warped integrated plate 1820 are visible. The right pedicle window 1835 of the inferior vertebral body 1825, along with the right 1840 and left 1845 pedicle windows of the superior vertebral body 1830 are shown as they would appear in an intra-operative x-ray. Note that the single superior level integral screw 1805 does not obstruct the right 1840 or left 1845 pedicle windows of the superior vertebral body 1830, which means that the device 1800 is ideal for use in conjunction with posterior instrumentation (rods and pedicle screws).

### Computer Implementation

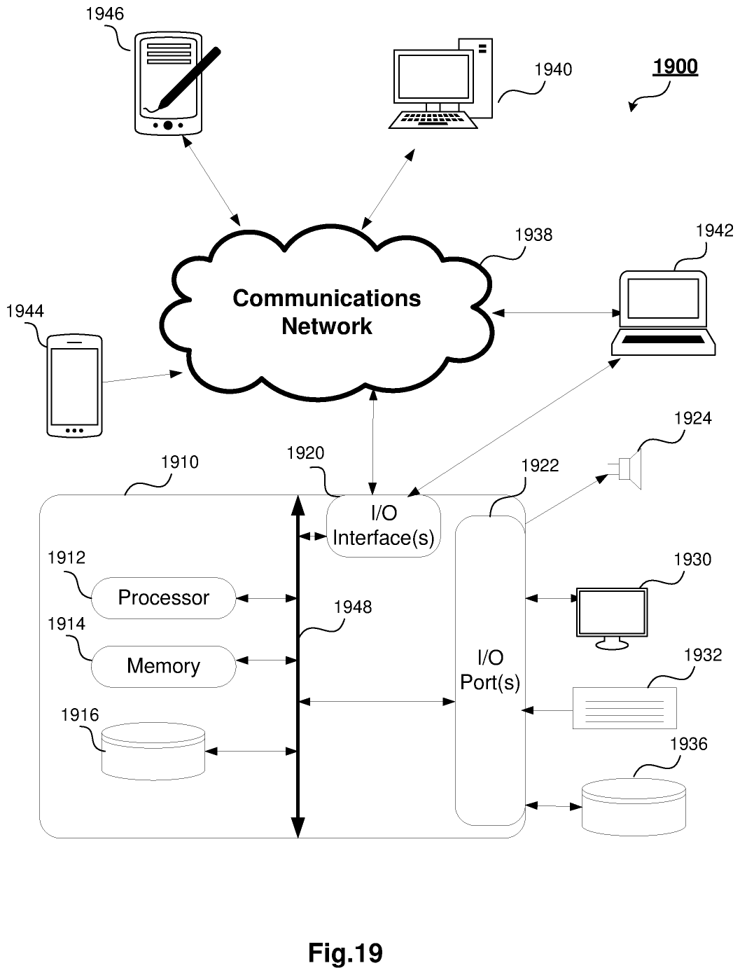

One or more methods of the present disclosure for producing medical devices utilise software executing on a computer. FIG. 19 is a schematic block diagram of a system 1900 that includes a general purpose computer 1910. The general purpose computer 1910 includes a plurality of components, including: a processor 1912, a memory 1914, a storage medium 1916, input/output (I/O) interfaces 1920, and input/output (I/O) ports 1922. Components of the general purpose computer 1910 generally communicate using one or more buses 1948.

The memory 1914 may be implemented using Random Access Memory (RAM), Read Only Memory (ROM), or a combination thereof. The storage medium 1916 may be implemented as one or more of a hard disk drive, a solid state “flash” drive, an optical disk drive, or other storage means. The storage medium 1916 may be utilised to store one or more computer programs, including an operating system, software applications, and data. In one mode of operation, instructions from one or more computer programs stored in the storage medium 1916 are loaded into the memory 1914 via the bus 1948. Instructions loaded into the memory 1914 are then made available via the bus 1948 or other means for execution by the processor 1912 to implement a mode of operation in accordance with the executed instructions.

One or more peripheral devices may be coupled to the general purpose computer 1910 via the I/O ports 1922. In the example of FIG. 19, the general purpose computer 1910 is coupled to each of a speaker 1924, a camera 1926, a display device 1930, an input device 1932, a printer 1934, and an external storage medium 1936. The speaker 1924 may be implemented using one or more speakers, such as in a stereo or surround sound system. In the example in which the general purpose computer 1910 is utilised to implement methods for producing patient-specific devices, one or more peripheral devices may relate to scanners or additive manufacturing machines connected to the I/O ports 1922.

The camera 1926 may be a webcam, or other still or video digital camera, and may download and upload information to and from the general purpose computer 1910 via the I/O ports 1922, dependent upon the particular implementation. For example, images recorded by the camera 1926 may be uploaded to the storage medium 1916 of the general purpose computer 1910. Similarly, images stored on the storage medium 1916 may be downloaded to a memory or storage medium of the camera 1926. The camera 1926 may include a lens system, a sensor unit, and a recording medium.

The display device 1930 may be a computer monitor, such as a cathode ray tube screen, plasma screen, or liquid crystal display (LCD) screen. The display 1930 may receive information from the computer 1910 in a conventional manner, wherein the information is presented on the display device 1930 for viewing by a user. The display device 1930 may optionally be implemented using a touch screen to enable a user to provide input to the general purpose computer 1910. The touch screen may be, for example, a capacitive touch screen, a resistive touchscreen, a surface acoustic wave touchscreen, or the like.

The input device 1932 may be a keyboard, a mouse, a stylus, drawing tablet, or any combination thereof, for receiving input from a user. The external storage medium 1936 may include an external hard disk drive (HDD), an optical drive, a floppy disk drive, a flash drive, solid state drive (SSD), or any combination thereof and may be implemented as a single instance or multiple instances of any one or more of those devices. For example, the external storage medium 1936 may be implemented as an array of hard disk drives.

The I/O interfaces 1920 facilitate the exchange of information between the general purpose computing device 1910 and other computing devices. The I/O interfaces may be implemented using an internal or external modem, an Ethernet connection, or the like, to enable coupling to a transmission medium. In the example of FIG. 19, the I/O interfaces 1922 are coupled to a communications network 1938 and directly to a computing device 1942. The computing device 1942 is shown as a personal computer, but may be equally be practised using a smartphone, laptop, or a tablet device. Direct communication between the general purpose computer 1910 and the computing device 1942 may be implemented using a wireless or wired transmission link.

The communications network 1938 may be implemented using one or more wired or wireless transmission links and may include, for example, a dedicated communications link, a local area network (LAN), a wide area network (WAN), the Internet, a telecommunications network, or any combination thereof. A telecommunications network may include, but is not limited to, a telephony network, such as a Public Switch Telephony Network (PSTN), a mobile telephone cellular network, a short message service (SMS) network, or any combination thereof. The general purpose computer 1910 is able to communicate via the communications network 1938 to other computing devices connected to the communications network 1938, such as the mobile telephone handset 1944, the touchscreen smartphone 1946, the personal computer 1940, and the computing device 1942.

One or more instances of the general purpose computer 1910 may be utilised to execute software to warp digital models of medical devices to have one or more surfaces that are contoured to match the anatomy of a patient. Such software may utilise computer instructions to perform the warping set out in the patent applications AU 2016900216, AU 2021902738, and PCT/AU2017/050056 or other warping implementations. In such embodiments, the memory 1914 and storage 1916 are utilised to store data relating to digital models, target points, source points, warping interpolation functions, and the like. Software for implementing the method for generating a model of a medical device is stored in one or both of the memory 1914 and storage 1916 for execution on the processor 1912, thus realising an improved computing device that is an advance in computer technology, as a computing device so programmed is capable of performing functionality not previously realised. The software includes computer program code for implementing method steps in accordance with the methods described herein of producing a digital models to be used in the production of integrally formed medical devices.

### FURTHER EMBODIMENTS

One embodiment provides a medical device comprising: a body portion; a graft window defining a vertical aperture through said body portion; and an alignment aid defining an aperture from an external wall of said body portion to an inner wall of said graft window.

In some implementations, medical device is integrally formed using an additive manufacturing process.

In some implementations, the alignment aid is asymmetric in shape.

In some implementations, the alignment aid is a triangle window.

In some implementations, the medical device further includes a set of structural members spanning the aperture. The set of structural members may form a lattice or array of structural members. The structural members may be located at the openings of the aperture, within the aperture, or a combination thereof. Such a set of structural members provides increased surface area for bone on-growth and allow the aperture to be larger in size, thus reducing the overall volume of material in the device.

A further embodiment provides a medical device comprising: a body portion; a graft window defining a vertical aperture through said body portion; and a plurality of alignment aids, each alignment aid defining an aperture from an external wall of said body portion to an inner wall of said graft window.

In some implementations, a pair of alignment aids are formed in opposing walls of the body portion.

A yet further embodiment provides a medical device comprising: a body portion; a graft window defining a vertical aperture through said body portion; and a set of anti-expulsion teeth defining a plurality of linear channels in an exterior surface of said body portion.

In some implementations, the medical device is integrally formed using an additive manufacturing process.

In some implementations, the medical device is a patient-specific device, wherein a region of said exterior surface of said body portion in which said anti-expulsion teeth are located is contoured to fit the anatomy of a patient.

### INDUSTRIAL APPLICABILITY

The arrangements described are applicable to the medical and healthcare industries.

The foregoing describes only some embodiments of the present invention, and modifications and/or changes can be made thereto without departing from the scope and spirit of the invention, the embodiments being illustrative and not restrictive.

Reference throughout this specification to “one embodiment”, “an embodiment,” “some embodiments”, or “embodiments” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

While some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practised without these specific details. In other instances, well-known methods, structures and techniques have not been shown in detail in order not to obscure an understanding of this description.

Note that when a method is described that includes several elements, e.g., several steps, no ordering of such elements, e.g., of such steps, is implied, unless specifically stated.

In the context of this specification, the word “comprising” and its associated grammatical constructions mean “including principally but not necessarily solely” or “having” or “including”, and not “consisting only of”. Variations of the word “comprising”, such as “comprise” and “comprises” have correspondingly varied meanings.

Similarly, it is to be noticed that the term coupled should not be interpreted as being limitative to direct connections only. The terms “coupled” and “connected,” along with their derivatives, may be used. It should be understood that these terms are not intended as synonyms for each other but may be. Thus, the scope of the expression “a device A coupled to a device B” should not be limited to devices or systems wherein an input or output of device A is directly connected to an output or input of device B. It means that there exists a path between device A and device B which may be a path including other devices or means in between. Furthermore, “coupled to” does not imply direction. Hence, the expression “a device A is coupled to a device B” may be synonymous with the expression “a device B is coupled to a device A”. “Coupled” may mean that two or more elements are either in direct physical or electrical contact, or that two or more elements are not in direct contact with each other but yet still co-operate or interact with each other.

As used throughout this specification, unless otherwise specified, the use of ordinal adjectives “first”, “second”, “third”, “fourth”, etc., to describe common or related objects, indicates that reference is being made to different instances of those common or related objects, and is not intended to imply that the objects so described must be provided or positioned in a given order or sequence, either temporally, spatially, in ranking, or in any other manner.

Although the invention has been described with reference to specific examples, it will be appreciated by those skilled in the art that the invention may be embodied in many other forms.

1. An integrally formed medical device, wherein said medical device is integrally formed using an additive manufacturing process, said medical device comprising:
a substantially trapezoidal body portion having at least one surface that is contoured to fit an anatomy of a patient;
a graft window defining a vertical aperture through said body portion, such that said body portion defines a cage; and
a radiographic alignment aid defining an aperture from an external wall of said body portion to an inner wall of said graft window.

2. (canceled)

3. The medical device according to claim 1, wherein said body portion has a superior surface contoured to fit the anatomy of the patient.

4. The medical device according to claim 1, further comprising:
an identifier, wherein said identifier is one of debossed or embossed on a surface of said body portion.

5. (canceled)

6. (canceled)

7. (canceled)

8. The medical device according to claim 1, wherein said alignment aid is an asymmetric shape indicating the correct orientation of the medical device.

9. The medical device according to 8, wherein said alignment aid is a triangle window, with an upper vertex indicating the top of the medical device.

10. The medical device according to claim 1, further comprising:
at least one insertion hole configured to receive a fixation screw at a predefined angle.

11. The medical device according to claim 10, wherein at least one of said at least one insertion holes is integrally formed as being threaded.

12. The medical device according to claim 10, wherein said medical device includes a pair of insertion holes in an anterior face of said body portion, the pair of insertion holes being offset relative to each other at a predefined angle.

13. The medical device according to claim 1, further comprising:
a threaded screw hole in face of said body portion, said threaded screw hole adapted to receive one of a threaded medical device insertion instrument and a locking cap.

14. The medical device according to claim 13, wherein said threaded screw hole is positioned such that when a locking cap is received in said screw hole, the locking cap covers at least a portion of at least one of said insertion holes.

15. The medical device according to claim 1, further comprising:
a set of anti-expulsion teeth defining a plurality of linear channels in an exterior surface of said body portion.

16. (canceled)

17. The medical device according to claim 1, wherein said medical device is a spinal implant device selected from the group consisting of: anterior cervical discectomy and fusion (ACDF) device, anterior lumbar interbody fusion (ALIF) device, cervical corpectomy device, lumbar corpectomy device, or lateral approach device.

18. The medical device according to claim 1, further comprising:
a plate abutting said body region.

19. The medical device according to claim 18, wherein an exterior surface of said plate is contoured to fit the anatomy of the patient.

20. The medical device according to claim 18, wherein said plate is in-line with a predominant load transmission pathway and is configured to increase a contact surface area between the device and the anatomy of the patient; or
perpendicular to a predominant load transmission pathway and is configured to act as an insertion stop governing the depth of insertion of the device.

21. The medical device according to claim 18, further comprising at least one buttress supports between said body portion and said plate.

22. (canceled)

23. The medical device according to claim 18, wherein said plate includes at least one integral screw hole offset from a plane of said body portion.

24. A method of producing an integrally formed patient-specific medical device, comprising the steps of:
importing a first digital model of a medical device to fit an anatomy of a patient, wherein said medical device has a substantially trapezoidal body portion and a graft window defines a vertical aperture through said body portion, such that said body portion defines a cage;
warping at least one surface of said first digital model of said medical device to produce a second digital model of a patient-specific device;
amending said second digital model of a patient-specific device to incorporate a radiographic alignment aid defining an aperture from an external wall of said body portion to an inner wall of said graft window; and
utilising an additive manufacturing machine to manufacture said integrally formed patient-specific medical device, based on the second digital model of the device.

25. The method according to 24, wherein said first digital model of said medical device is a triangulated vertex boundary representation suitable for 3D printing, and wherein said warping is performed by:
importing a third digital model of a target shape associated with the anatomy of the patient;
determining a warping interpolation function based on relative positions of a set of source points associated with the first digital model and relative positions of the same points projected to the target shape; and
applying the warping interpolation function to all vertices of the medical device to generate said second digital model of said device.

26. (canceled)

27. (canceled)

28. The medical device according to claim 1, wherein the radiographic alignment aid is a subtractive design radiographic alignment aid.
